(19) United States
(12) Patent Application Publication
(10) Pub. No.: US 2011/0104273 A1
(43) Pub. Date: May 5, 2011

(54) GASTRIC RETENTIVE PHARMACEUTICAL COMPOSITIONS FOR IMMEDIATE AND EXTENDED RELEASE OF PHENYLEPHRINE

(75) Inventor: Sui Yuen Eddie Hou, Foster City, CA (US)

(73) Assignee: DEPOMED, INC., Menlo Park, CA (US)

(21) Appl. No.: 12/940,964

(22) Filed: Nov. 5, 2010

Related U.S. Application Data
(60) Provisional application No. 61/258,460, filed on Nov. 5, 2009.

(51) Int. Cl. A61K 31/137; A61K 9/14; A61K 9/26; A61P 11/02; A61P 27/02; A61P 9/00; A61K 9/28

(52) U.S. Cl. 424/465; 424/484; 424/487; 424/486; 424/488; 424/485; 424/469; 514/653; 427/2.14

(57) ABSTRACT

Gastric retentive dosage forms for both immediate and extended release of phenylephrine are described which allow once- or twice-daily dosing. Methods of treatment using the dosage forms and methods of making the dosage forms are also described.

### CROSS-REFERENCE TO RELATED APPLICATIONS

This application claims the benefit of U.S. Provisional Application No. 61/258,460, filed Nov. 5, 2009, which is incorporated herein by reference in its entirety.

### TECHNICAL FIELD

The present subject matter relates generally to a dosage form for both immediate release and extended release of phenylephrine into the stomach of a patient in the fed mode and to methods of treatment using the dosage forms.

### BACKGROUND

Phenylephrine (PE) is used as a decongestant sold as an oral medicine, as a nasal spray, or as eye drops. Phenylephrine has become the most common over-the-counter (OTC) decongestant in the United States as a result of greater restrictions placed on pseudoephedrine (PDE) due to its use a methamphetamine precursor.

PE and PDE are sympathomimetic vasoconstrictors that are closely related to adrenaline in structure. PE differs chemically from adrenaline only in the absence of one hydroxyl group from the benzene ring. It is used commercially as the (−)-enantiomer as PE hydrochloride. Oral phenylephrine is extensively metabolised by monoamine oxidase, an enzyme that is present in the gastrointestinal tract and in the liver. Therefore, compared to orally-taken pseudoephedrine, it has a reduced and variable bioavailability of only up to 38 percent,[3][4]. Because phenylephrine is a direct selective α-adrenergic receptor agonist, it does not cause the release of endogenous noradrenaline, as pseudoephedrine does. Therefore, phenylephrine is less likely to cause side-effects such as central nervous system stimulation, insomnia, anxiety, irritability, and restlessness.

Phenylephrine, is a relatively selective α1-adrenergic receptor agonist used primarily as a decongestant, as an agent to dilate the pupil, and to increase blood pressure. Phenylephrine has recently been marketed as a substitute for pseudoephedrine (e.g., Pfizer's Sudafed (Original Formulation)).

Phenylephrine is well absorbed from the gut. However, after oral administration, phenylephrine is subject to extensive presystemic metabolism by monoamine oxidase in the gut wall. As a consequence of metabolism, systemic bioavailability of PE is only around 40%. Only about 3% of an oral dose of PE is excreted unchanged in the urine. The efficacy of phenylephrine formulated as a topical nasal decongestant nasal spray (0.25-0.5% w/v) is supported by several studies.

In oral over-the-counter (OTC) doses, phenylephrine has minimal effects on the cardiovascular system. When administered intravenously, phenylephrine causes an increase in arterial blood pressure and bradycardia and may also cause coronary vasospasm. The threshold dosage OTC phenylephrine administered orally in man for any effects on the cardiovascular system is about 50 mg and at this dose PE causes a decline in heart rate and a slight increase in arterial blood pressure. Thus, there is clearly a need for a controlled-release dosage form for delivery of phenylephrine in both a safe and an effective manner.

Gastric retained oral dosage forms are one approach for delivery of drugs in the upper portions of the gastrointestinal (GI) tract, and have been previously described, for example, in Gusler et al. (U.S. Pat. No. 6,723,34), Berner et al. (U.S. Pat. No. 6,488,962), Shell et al., (U.S. Pat. No. 6,340,475) and Shell et al. (U.S. Pat. No. 6,635,280). These formulations make use of one or more hydrophilic polymers that swell upon intake of water from gastric fluid. When administered to a subject in the fed mode, when the size of pyloric sphincter is reduced, the dosage form swells to a size effective for its retention in the stomach for a minimum of about four hours.

Successful formulation of a gastric retentive dosage form for any given drug requires careful design and selection of the formulation components. For example, the gastric retentive dose for requires an amount of swellable polymer such that upon administration, it will swell to a size sufficient for gastric retention. However, too much swellable polymer will result in a pill too large to swallow. Too little polymer will result in insufficient swelling such that the pill escapes through the pylorus too soon. Additionally, the dosage form must contain enough of the pharmaceutically active agent to maintain desired levels in the blood, thereby providing therapeutic efficacy over the desired period of time, for example, about 12 hours.

### BRIEF SUMMARY

The present disclosure provides, among other aspects, gastric retentive dosage forms for oral administration to a subject, such as a human patient, for relief from symptoms including, but not limited to, ophthalmic disorders (hyperaemia of conjunctiva, posterior synechiae, acute atopic), nasal congestion, hemorrhoids, hypotension, shock, hypotension during spinal anesthesia, and paroxysmal supraventricular tachycardia. The dosage form in some embodiments is a gastric retentive dosage form that contains a first dose of phenylephrine as an immediate release (“IR”) layer, and a second dose of phenylephrine as an extended release (“ER”) layer.

In one aspect, the ER layer of the dosage form comprises the second dose of phenylephrine dispersed in a polymer matrix comprising at least one hydrophilic polymer. Upon administration, the polymer matrix swells upon imbibition of fluid to a size sufficient such that the ER portion of the dosage form is retained in a stomach of a subject in a fed mode and the second dose of phenylephrine is released over an extended period of time.

In one embodiment, the ER layer comprises a hydrophilic polymer having an average molecular weight ranging from about 200,000 Da (Daltons) to about 7,000,000 Da, about 900,000 Da to about 5,000,000 Da, about 2,000,000 Da to about 5,000,000 Da, from about 4,000,000 Da to about 5,000,000 Da, from about 2,000,000 Da to about 4,000,000 Da, from about 900,000 Da to about 5,000,000 Da, or from about 900,000 Da to about 4,000,000 Da. In another embodiment, the ER layer comprises a hydrophilic polymer having an average molecular weight of equal to or greater than about 200,000 Da, 600,000 Da, 900,000 Daltons, 1,000,000 Da, 2,000,000 Da, 4,000,000 Da, 5,000,000 Da, or 7,000,000 Da.

In one embodiment, the ER layer comprises a hydrophilic polymer having an average viscosity ranging from about 4,000 cp to about 200,000 cp (centipoise), from about 50,000 cp to about 200,000 cp, or from about 80,000 cp to about 120,000 cp, as measured as a 2% weight per volume in water at 20° C.

In one embodiment, the ER layer comprises a total amount of hydrophilic polymer which is between about 100 mg and 225 mg (milligrams) or about 125 mg to about 200 mg. In another embodiment, the total amount of hydrophilic polymer in the ER layer is about 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, or 190 mg. In yet another embodiment, the total amount of hydrophilic polymer in the ER layer is present in an amount which is about 50 wt % to about 40 wt %, about 20 wt % to about 40 wt % or about 10 wt % to about 30 wt % (weight percent) of the ER layer. In yet another embodiment, the total amount of hydrophilic polymer in the ER layer is present in an amount which is about 10 wt %, 12 wt %, 14 wt %, 15 wt %, 17 wt %, 18 wt %, 20 wt %, 22 wt %, 24 wt %, 25 wt %, 27 wt %, 30 wt %, 35 wt % or 40 wt % of the ER layer.

In one embodiment, the at least one hydrophilic polymer in the ER layer is cellulose, an alkyl-substituted cellulose, a crosslinked polyacrylic acid, or a xanthan gum. In another embodiment, the alkyl-substituted cellulose is hydroxymethyl-cellulose, hydroxyethyl-cellulose, hydroxypropyl-cellulose, hydroxypropyl methylcellulose, or carboxymethyl-cellulose. In still another embodiment, the hydroxypropyl methylcellulose has a viscosity ranging from 11,000 to 110,000 centipoise as measured in a 2% solution at 20° C.

In one embodiment, the at least one hydrophilic polymer in the ER layer is a polyalkylene oxide. In another embodiment, the at least one hydrophilic polymer is poly(ethylene oxide). In yet another embodiment, the at least one hydrophilic polymer in the ER layer is a cellulose.

In one embodiment, the ER layer comprises two swellable hydrophilic polymers in a ratio of 3:1, 3:1.5, 3:2, 2:1, 2:1.5, 1:1, 1:1.5, 1:2, 1:2.5, or 1:3.

In one embodiment, the ER layer comprises between about 5 mg to about 40 mg of phenylephrine. In another embodiment, the ER layer comprises about 5 mg, 10 mg, 15 mg, 20 mg, 25 mg, 30 mg, 35 mg, 40 mg, 45 mg, or 50 mg phenylephrine.

In one embodiment, the ER layer comprises phenylephrine that is present in an amount that is between about 3 wt % to about 15 wt % of the ER layer. In another embodiment, the ER layer comprises phenylephrine that is present in an amount that is about 3 wt %, 4 wt %, 5 wt %, 6 wt %, 7 wt %, 8 wt %, 9 wt %, 10 wt %, 11 wt % 12 wt %, 13 wt %, 14 wt %, or 15 wt % of the ER layer.

In one embodiment, the ratio of phenylephrine to hydrophilic polymer in the ER layer ranges from about 9:1 to about 4:1. In another embodiment, the ratio of phenylephrine to hydrophilic polymer in the ER layer is about 9:1, 8:1, 7:1, 6:1, 5:1, or 4:1.

In one embodiment, the phenylephrine is released from the ER layer over a time period of 6 h to 11 h (hours), 6 h to 9 h, 7 h to 9 h, 8 h to 9 h, 8 h to 10 h, or 9 h to 10 h. In another embodiment, phenylephrine is delivered to the small intestine of the subject over a time period of at least 1 h, 2 h, 3 h, 4 h, 5 h, 6 h, 7 h, 8 h, 9 h, 10 h, 11 h, or 12 h.

In one embodiment, the phenylephrine is released from the ER layer via diffusion. In another embodiment, the phenylephrine is released from the ER layer via erosion. In yet another embodiment, the phenylephrine is released from the ER layer via a combination of diffusion and erosion.

In one embodiment, the ER layer comprises a binder which is polyvinylpyrrolidone, hydroxypropylcellulose (HPC), microcrystalline cellulose (MCC), polyvinylalcohol, ethyl cellulose, lactose, or polyethylene glycol. In yet another embodiment, the polyvinylpyrrolidone is povidone, copovidone, or crospovidone. In yet another embodiment, the ER layer comprises a combination of more than one binder.

In one embodiment, the ER layer further comprises one or more binders. In another embodiment, the one or more binders is in an amount ranging from about 15 mg to about 80 mg. In another embodiment, the total amount of the one or more binder in the ER layer is about 15 mg, 17 mg, 19 mg, 20 mg, 21 mg, 23 mg, 25 mg, 27 mg, 30 mg, 32 mg, 34 mg, 35 mg, 37 mg, 39 mg, 40 mg, 45 mg, 47 mg, 50 mg, 55 mg, 57 mg, 60 mg, 64 mg, 65 mg, 67 mg, 70 mg, 75 mg, or 80 mg. In yet another embodiment, the amount of the one or more binder in the ER layer is about 2.5 mg, 2.7 mg, 3.0 mg, 3.2 mg, 3.5 mg, 3.7 mg, 4.0 mg, 4.3 mg, 4.5 mg, 4.7 mg, 5.0 mg, 5.3 mg, 5.5 mg, 5.7 mg, 6.0 mg, 6.5 mg, 7.0 mg, 7.5 mg, 8.0 mg, 8.5 mg, 9.0 mg, 9.5 mg, or 10.0 wt % of the ER layer.

In one embodiment, the ER layer further comprises a lubricant which is magnesium stearate, calcium stearate, sodium stearyl fumarate, stearic acid, stearyl behenate, glyceryl behenate, or polyethylene glycol.

In one embodiment, the ER layer comprises one or more lubricants which is present in an amount ranging from about 0.3 mg to 10 mg. In yet another embodiment, the amount of the one or more lubricants in the ER layer is about 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg or 10 mg. In yet another embodiment, the amount of the one or more lubricants in the ER layer is about 0.4 wt %, 0.5 wt %, 0.6 wt %, 0.7 wt %, 0.8 wt %, 0.9 wt %, 1.0 wt %, 1.2 wt %, 1.4 wt %, 1.6 wt %, 1.8 wt %, 2.0 wt %, 2.2 wt %, 2.4 wt %, or 2.5 wt % of the ER layer.

In one embodiment, the ER layer comprises an anti-oxidant which is ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, or propylgallate. In another embodiment, the antioxidant is present in the dosage for at a wt % (weight percent) of approximately 0.01 wt %, 0.05 wt %, 0.1 wt %, 0.5 wt %, 0.75 wt %, 1 wt %, 2 wt %, 3 wt % or 4 wt %.

In one embodiment, the ER layer comprises a chelating agent which is ethylenediamine tetracetic acid (EDTA) and its salts, ethylene glycol tetraacetic acid (EGTA) and its salts, dihydroxy ethyl glycine, citric acid or tartaric acid. In another embodiment, the chelating agent is present in the dosage for at a wt % of approximately 0.01 wt %, 0.05 wt %, 0.1 wt %, 0.5 wt %, 0.75 wt %, 1 wt %, 2 wt %, 3 wt % or 4 wt %.

In one embodiment, the ER layer comprises one or more additional excipients which are diluents, coloring agents, flavoring agents, and/or glidants.

In one embodiment, the dosage form further comprises an IR layer which comprises a first dose of phenylephrine. In another embodiment, the first dose of phenylephrine is between about 5 mg to about 20 mg or between about 10 mg to about 15 mg phenylephrine. In another embodiment, the IR layer comprises about 5 mg, 6 mg, 7 mg, 7.5 mg, 8 mg, 9 mg, 10 mg, 11 mg, 12 mg, 13 mg, 14 mg or 15 mg phenylephrine. In yet another embodiment, the IR layer comprises a first dose of phenylephrine which is present in an amount which is about 5 wt % to about 15 wt % of the IR layer. In yet another embodiment, the first dose of phenylephrine is present in an amount which is about 5 wt %, 6 wt %, 7 wt %, 8 wt %, 9 wt %, 10 wt %, 11 wt %, 12 wt %, 13 wt %, 14 wt %, or 15 wt % of the IR layer.

In one embodiment, the IR layer further comprises a binder. In another embodiment, the binder is polyvinylpyrrolidone (PVP), polyvinylalcohol, hydroxypropyl cellulose (HPC), microcrystalline cellulose (MCC) ethyl cellulose, or polyethylene glycol. In yet another embodiment, the polyvinylpyrrolidone is povidone, copovidone, or crospovidone. In yet another embodiment, the IR layer comprises a combination of more than one binder.

In one embodiment, the IR layer may comprise a binder in an amount ranging from about 6 mg to about 50 mg or from about 6 mg to about 12 mg. In another embodiment, the total amount of the binder in the IR layer is about 5 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 15.0 mg, 17.0 mg, 19.0 mg, 20.0 mg, 23.0 mg, 25.0 mg, 27.0 mg, 30.0 mg, 33.0 mg, 35.0 mg, 37.0 mg, 40.0 mg, 45.0 mg, 47.0 mg, 50.0 mg, 55.0 mg, 57.0 mg, or 60.0 mg. In yet another embodiment, the amount of binder in the IR layer is in an amount which is about 2.5 wt %, 2.7 wt %, 3.0 wt %, 3.2 wt %, 3.5 wt %, 3.7 wt %, 4.0 wt %, 4.3 wt %, 4.5 wt %, 4.7 wt %, 5.0 wt %, 5.3 wt %, 5.5 wt %, 5.7 wt %, 6.0 wt %, 6.5 wt %, 7.0 wt %, 7.5 wt %, 8.0 wt %, 8.5 wt %, 9.0 wt %, 9.5 wt %, or 10.0 wt % of the IR layer.

In one embodiment, the IR layer comprises a disintegrant which is cellulose or a derivative of cellulose such as microcrystalline cellulose, crosspovidone, crosslinked starch such as sodium starch glycolate, alginic acid or soy polysaccharides.

In one embodiment, the IR layer comprises a lubricant which is magnesium stearate, calcium stearate, sodium stearyl fumarate, stearic acid, stearyl behenate, glyceryl behenate, or polyethylene glycol.

In one embodiment, the IR layer comprises an anti-oxidant which is ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, or propylgallate. In another embodiment, the antioxidant is present in the IR layer at a wt % (weight percent) of approximately 0.01 wt %, 0.05 wt %, 0.1 wt %, 0.5 wt %, 0.75 wt %, 1 wt %, 2 wt %, 3 wt % or 4 wt %.

In one embodiment, the IR layer comprises a lubricant which is present in an amount ranging from about 0.2 mg to about 10.0 mg or from about 1.0 mg to about 10.0 mg. In yet another embodiment, the amount of lubricant in the IR layer is about 0.2 mg, 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 or 10 mg. In yet another embodiment, the amount of lubricant in the IR layer is about 0.4 wt %, 0.5 wt %, 0.6 wt %, 0.7 wt %, 0.8 wt %, 0.9 wt %, 1.0 wt %, 1.2 wt %, 1.4 wt %, 1.6 wt %, 1.8 wt %, 2.0 wt %, 2.1 wt %, 2.2 wt %, 2.4 wt %, or 2.5 wt % of the IR layer.

In one embodiment, the IR layer comprises a chelating agent which is ethylenediamine tetracetic acid (EDTA) and its salts, ethylene glycol tetraacetic acid (EGTA) and its salts, dihydroxy ethyl glycine, citric acid or tartaric acid. In another embodiment, the chelating agent is present in the dosage for at a wt % of approximately 0.01 wt %, 0.05 wt %, 0.1 wt %, 0.5 wt %, 0.75 wt %, 1 wt %, 2 wt %, 3 wt % or 4 wt %.

In one embodiment, a gastric retentive dosage form comprising an IR layer with a first dose of phenylephrine and an ER layer with a second dose of phenylephrine is provided. In another embodiment, the first dose of phenylephrine is about 5 mg, 6 mg, 7 mg, 8 mg, 9 mg, 10 mg, 11 mg, 12 mg, 13 mg, 14 mg, or 15 mg and the second dose of phenylephrine is about 5 mg, 10 mg, 15 mg, 20 mg, 25 mg, 30 mg, 35 mg, 40 mg, 45 mg, or 50 mg. In yet another embodiment, the total dose of phenylephrine in the gastric retentive dosage form is about 10 mg, 15 mg, 20 mg, 25 mg, 30 mg, 35 mg, 40 mg, 45 mg, 50 mg, 55 mg, or 60 mg.

In one embodiment, the gastric retentive dosage is a tablet. In another embodiment, the tablet has a total weight ranging from about 200 mg to about 1000 mg, or from about 400 mg to about 800 mg or from about 500 mg to about 700 mg. In yet another embodiment, the tablet has a total weight of about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg.

In one embodiment, the dosage form is a pharmaceutical tablet, such as a gastric retentive tablet for the extended release of phenylephrine. In another embodiment, the tablet is a monolithic tablet comprising an ER layer. In another embodiment, the tablet is a monolithic tablet comprising an ER layer and an IR layer. In another embodiment, the tablet is a bilayer tablet, comprising an ER layer and an IR layer. The bilayer tablet is typically a monolithic tablet. In another embodiment, the dosage form is a capsule comprising an ER layer. In another embodiment, the dosage form is a capsule comprising ER layer and an IR layer.

In some embodiments, the bilayer tablet has a friability of no greater than about 0.1%, 0.2% 0.3%, 0.4%, 0.5%, 0.7% or 1.0%.

In some embodiments, the bilayer tablet has a hardness of at least about 10 kilopond (also known as kilopons) (kp). In some embodiments, the tablet has a hardness of about 9 kp to about 25 kp, or about 12 kp to about 20 kp. In further embodiments, the tablet has a hardness of about 11 kp, 12 kp, 13 kp, 14 kp, 15 kp, 16 kp, 17 kp, 18 kp, 19 kp, 20 kp, 21 kp, 22 kp, 23 kp, 24 kp, or 25 kp.

In some embodiments, the tablets have a content uniformity of from about 85 to about 115 percent by weight or from about 90 to about 110 percent by weight, or from about 95 to about 105 percent by weight. In other embodiments, the content uniformity has a relative standard deviation (RSD) equal to or less than about 3.5%, 3.0%, 2.5%, 2.0%, 1.5%, 1.0% or 0.5%.

In one aspect, the phenylephrine in the oral dosage form is stable over a period of at least 1 month, 3 months, 5 months, 7 months, 9 months, 12 months, 15 months, 20 months, 24 months, 2.5 years or 3 years. In one embodiment, stable phenylephrine is defined as less than 0.1%, 0.2%, 0.3%, 0.5%, 0.75%, 1.0%, 1.5%, 1.75%, 2.0%, 2.5%, 3.0%, 3.5% or 4.0% degradation or oxidation of the dose of phenylephrine over a time period of at least 1 month, 3 months, 5 months, 7 months, 9 months, 12 months, 15 months, 20 months, 24 months, 2.5 years or 3 years. In another embodiment, stability is determined at a constant temperature of about 25° C., 30° C., 45° C., 50° C., 60° C., 65° C., 70° C., or 75° C. over a time period. In still another embodiment, stability is determined at a relative humidity (RH) of about 40% RH, 50% RH, 60% RH, 65% RH, 70% RH, 75% RH.

In one embodiment, about 90% to about 100% of the first dose of phenylephrine is released within 15 minutes, 30 minutes, 45 minutes or 60 minutes after oral administration.

In one embodiment, the ER layer swells upon imbibition of fluid from gastric fluid to a weight which is approximately 115%, 120%, 140%, 150%, 175%, 200%, 225%, 250%, 275%, 300%, 325%, 350%, 375%, 400% or 435% of the weight of the ER layer form prior to imbibition.

In one embodiment, the dosage form provides a dissolution profile wherein between about 40% to about 80%, about 30% to about 60%, about 35% to about 55% or about 40% to about 50% of the second dose of phenylephrine remains in the ER layer between about 1 and 2 hours after administration. In one embodiment, not more than 50% of the second dose of phenylephrine is released within about the first hour. In a further embodiment, not more than 45% or not more than 40% of the second dose of phenylephrine is released within about the first hour. In another embodiment, not more than 85% of the second dose of phenylephrine is released within about 4 hours. In yet another embodiment, not less than 50% is released after about 6 hours. In yet another embodiment, not less than 60% is released after about 6 hours.

In one embodiment, the second dose of phenylephrine is released over a time period of about 6 to 10, about 8 to 10, or about 9 to 10 hours in vitro. In another embodiment, the second dose of phenylephrine is released over a time period of about 7 hours, 8 hours, 9 hours, 10 hours or 11 hours in vitro. In another embodiment, at least 90% or 95% of the second dose of phenylephrine is released over a time period of about 7 hours, 8 hours, 9 hours, 10 hours or 11 hours in vitro.

In one aspect, a method of making a gastric retentive dosage form comprising phenylephrine and at least one hydrophilic polymer is provided.

In one embodiment, the method of making the dosage form comprises granulating phenylephrine powder with at least one hydrophilic polymer. In another embodiment, the granulating is fluid bed or high shear granulation. In another embodiment, the method comprises direct compression of the at least one hydrophilic polymer with a pregranulated phenylephrine composition. In yet another embodiment, the granulated phenylephrine composition contains phenylephrine granulated with starch or povidone.

In one embodiment, a gastric retentive dosage form comprising phenylephrine is made by dry blending the phenylephrine with the hydrophilic polymer and optionally with one or more excipients prior to compressing the blended mixture into a tablet.

In one embodiment, a gastric retentive dosage form comprising phenylephrine and made by the process of directly compressing a pregranulated phenylephrine with one or more hydrophilic polymers is provided.

In one embodiment, a gastric retained dosage form comprising phenylephrine and at least one swellable polymer is administered to a subject suffering from ophthalmic disorders (hyperaemia of conjunctiva, posterior synechiae, acute atopic), nasal congestion, hemorrhoids, hypotension, shock, hypotension during spinal anesthesia, and paroxysmal supraventricular tachycardia.

In one embodiment, a gastric retained dosage form is administered to a subject in a fed mode. In another embodiment, the dosage form is administered with a meal to a subject once in a 24 hour period. In other embodiments, the dosage form is administered with a meal to the subject twice in a 24 hour period. In yet another embodiment, the dosage form is administered with a meal to a subject once or twice in a 24 hour period for 2, 3, 4, 5, 6, 7, 8 or more days.

### BRIEF DESCRIPTION OF THE DRAWINGS

FIG. 1 is a graph comparing dissolution release profiles for gastric retentive (GR) dosage forms identified herein as GR Prototype I tablets.

FIG. 2 is a graph comparing dissolution release profiles for gastric retentive (GR) dosage forms identified herein as GR Prototype II tablets.

FIG. 3 is a graph showing swelling profiles for a GR prototype II Formula D tablet.

FIG. 4 is a graph showing dissolution and disintegration release profiles for a GR prototype II Formula D tablet.

FIG. 5 is a graph showing dissolution profiles for GR Prototype II Formula D tablets stored at 25° C. and 60% relative humidity.

FIG. 6 is a graph showing dissolution profiles for GR Prototype II Formula D tablets stored at 40° C. and 75% relative humidity.

FIG. 7 is a dissolution profile for gastric retentive (GR) dosage forms identified herein as Bi-layer Prototype III tablets.

FIGS. 8A-H illustrate simulated release profile for gastric retentive phenylephrine dosage forms.

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art.

All publications, patents and patent applications cited herein, whether supra or infra, are hereby incorporated by reference in their entirety.

### I. DEFINITIONS

It must be noted that, as used in this specification, the singular forms “a,” “an,” and “the” include plural referents unless the context clearly dictates otherwise.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

“Optional” or “optionally,” as used herein, means that the subsequently described element, component or circumstance may or may not occur, so that the description includes instances where the element, component, or circumstance occurs and instances where it does not.

The terms “subject,” “individual” or “patient” are used interchangeably herein and refer to a vertebrate, preferably a mammal. Mammals include, but are not limited to, humans.

The term “about,” particularly in reference to a given quantity, is meant to encompass deviations of plus or minus five percent.

The gastric retentive oral dosage forms presented herein provide an immediate and extended release dose of phenylephrine that is released into the stomach of a subject in a fed mode.

The term “fed mode,” as used herein, refers to a state which is typically induced in a patient by the presence of food in the stomach, the food-giving rise to two signals, one that is said to stem from stomach distension and the other a chemical signal based on food in the stomach. It has been determined that once the fed mode has been induced, larger particles are retained in the stomach for a longer period of time than smaller particles; thus, the fed mode is typically induced in a patient by the presence of food in the stomach. The fed mode is initiated by nutritive materials entering the stomach upon the ingestion of food. Initiation is accompanied by a rapid and profound change in the motor pattern of the upper GI tract, over a period of 30 seconds to one minute. The change is observed almost simultaneously at all sites along the GI tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 6 hours. Administration of a dosage form “with a meal,” as used herein, refers to administration before, during or after a meal, and more particularly refers to administration of a dosage form about 1, 2, 3, 4, 5, 10, 15 minutes before commencement of a meal, during the meal, or about 1, 2, 3, 4, 5, 10, 15 minutes after completion of a meal.

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid. The specific results of dissolution tests claimed herein are performed on dosage forms or pharmaceutical compositions in a USP Type II apparatus and immersed in 900 ml of simulated intestinal fluid (SIF) at pH 6.8 and equilibrated in a constant temperature water bath at 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer” as used herein refers to a molecule containing a plurality of covalently attached monomer units, and includes branched, dendrimeric, and star polymers as well as linear polymers. The term also includes both homopolymers and copolymers, e.g., random copolymers, block copolymers and graft copolymers, as well as uncrosslinked polymers and slightly to moderately to substantially crosslinked polymers, as well as two or more interpenetrating cross-linked networks.

The term “swellable polymer,” as used herein, refers to a polymer that will imbibe a fluid, preferably water, and become enlarged or engorged. A polymer is swellable due, at least in part, to a structural feature of the polymer. Whether or not a swellable polymer when incorporated into a dosage form or matrix containing other components swells in the presence of fluid will depend upon a variety of factors, including the specific type of polymer and the percentage of that polymer in a particular formulation. For example, the term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights are: POLYOX® NF, grade WSR coagulant, approximate molecular weight 5 million, POLYOX® grade WSR 301, approximate molecular weight 4 million, POLYOX® grade WSR 303, approximate molecular weight 7 million, POLYOX® grade WSR N-60K, approximate molecular weight 2 million, and POLYOX® grade N-80K, approximate molecular weight 200,000. An oral dosage form which comprises a swellable polymer as used herein intends that the polymer when incorporated into the dosage form will swell upon imbibition of water or fluid from gastric fluid.

The terms “swellable” and “bioerodible” (or simply “erodible”) are used to refer to the polymers used in the present dosage forms, with “swellable” polymers being those that are capable of absorbing water and physically swelling as a result, with the extent to which a polymer can swell being determined by the molecular weight or degree of crosslinking (for crosslinked polymers), and “bioerodible” or “erodible” polymers referring to polymers that slowly dissolve and/or gradually hydrolyze in an aqueous fluid, and/or that physically disentangle or undergo chemical degradation of the chains themselves, as a result of movement within the stomach or GI tract.

The term “friability,” as used herein, refers to the ease with which a tablet will break or fracture. The test for friability is a standard test known to one skilled in the art. Friability is measured under standardized conditions by weighing out a certain number of tablets (generally 20 tablets or less), placing them in a rotating Plexiglas drum in which they are lifted during replicate revolutions by a radial lever, and then dropped approximately 8 inches. After replicate revolutions (typically 100 revolutions at 25 rpm), the tablets are reweighed and the percentage of formulation abraded or chipped is calculated. The friability of the tablets, of the present invention, is preferably in the range of about 0% to 3%, and values about 1%, or less, are considered acceptable for most drug and food tablet contexts. Friability which approaches 0% is particularly preferred.

The term “tap density” or “tapped density,” as used herein, refers to a measure of the density of a powder. The tapped density of a pharmaceutical powder is determined using a tapped density tester, which is set to tap the powder at a fixed impact force and frequency. Tapped density by the USP method is determined by a linear progression of the number of taps.

The term “bulk density,” as used herein, refers to a property of powders and is defined as the mass of many particles of the material divided by the total volume they occupy. The total volume includes particle volume, inter-particle void volume and internal pore volume.

The term “capping,” as used herein, refers to the partial or complete separation of top or bottom crowns of the tablet main body. For multilayer tablets, capping refers to separation of a portion of an individual layer within the multilayer tablet. Unintended separation of layers within a multilayer tablet prior to administration is referred to herein as “splitting.”

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

The terms “effective amount” or a “therapeutically effective amount” refer to the amount of drug or pharmacologically active agent to provide the desired effect without toxic effects. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. The term “pharmacologically active” (or simply “active”) as in a “pharmacologically active” derivative, refers to a derivative having the same type of pharmacological activity as the parent compound and/or drug and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA, or comparable agency.

The terms “drug,” “active agent,” “therapeutic agent,” and/or “pharmacologically active agent” are used interchangeably herein to refer to any chemical compound, complex or composition that is suitable for oral administration and that has a beneficial biological effect, preferably a therapeutic effect in the treatment or prevention of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc.

The term “dosage form” refers to the physical formulation of the drug for administration to the patient. Dosage forms include without limitation, tablets, capsules, caplets, liquids, syrups, lotions, lozenges, aerosols, patches, enemas, oils, ointments, pastes, powders for reconstitution, sachets, solutions, sponges, and wipes. Within the context of the present invention, a dosage form comprising an phenylephrine formulation will generally be administered to patients in the form of tablets or capsules, although a liquid formulation is also contemplated within this disclosure.

The term “dosage unit” refers to a single unit of the dosage form that is to be administered to the patient. The dosage unit will be typically formulated to include an amount of drug sufficient to achieve a therapeutic effect with a single administration of the dosage unit although where the size of the dosage form is at issue, more than one dosage unit may be necessary to achieve the desired therapeutic effect. For example, a single dosage unit of a drug is typically, one tablet, one capsule, or one tablespoon of liquid. More than one dosage unit may be necessary to administer sufficient drug to achieve a therapeutic effect where the amount of drug causes physical constraints on the size of the dosage form.

“Delayed release” dosage forms are a category of modified release dosage forms in which the release of the drug is delayed after oral administration for a finite period of time after which release of the drug is unhindered. Delayed release dosage forms are frequently used to protect an acid-labile drug from the low pH of the stomach or where appropriate to target the GI tract for local effect while minimizing systemic exposure. Enteric coating is frequently used to manufacture delayed release dosage forms.

The terms “sustained release,” and “extended release” are used interchangeably herein to refer to a dosage form that provides for release of a drug over an extended period of time. With extended release dosage forms, the rate of release of the drug from the dosage form is reduced in order to maintain therapeutic activity of the drug for a longer period of time or to reduce any toxic effects associated with a particular dosing of the drug. Extended release dosage forms have the advantage of providing patients with a dosing regimen that allows for less frequent dosing, thus enhancing compliance. Extended release dosage forms can also reduce peak-related side effects associated with some drugs and can maintain therapeutic concentrations throughout the dosing period thus avoiding periods of insufficient therapeutic plasma concentrations between doses.

The term “modified release” refers to a dosage form that includes both delayed and extended release drug products. The manufacture of delayed, extended, and modified release dosage forms are known to ordinary skill in the art and include the formulation of the dosage forms with excipients or combinations of excipients necessary to produce the desired active agent release profile for the dosage form.

The “gastric retentive” oral dosage forms described herein are a type of extended release dosage form. Gastric retentive dosage forms are beneficial for the delivery of drugs with reduced absorption in the lower GI tract or for local treatment of diseases of the stomach or upper GI tract. For example, in certain embodiments of gastric retentive oral dosage forms of the present invention, the dosage form swells in the gastric cavity and is retained in the gastric cavity of a patient in the fed med so that the drug may be released for heightened therapeutic effect. See, Hou et al., Crit. Rev. Ther. Drug Carrier Syst. 20(6):459-497 (2003).

The in vivo “release rate” and in vivo “release profile” refer to the time it takes for the orally administered dosage form, or the active agent-containing layer of a bilayer or multilayer tablet (administered when the stomach is in the fed mode) or the content of the active ingredient to be reduced to 0-10%, preferably 0-5%, of its original size or level, as may be observed visually using NMR shift reagents or paramagnetic species, radio-opaque species or markers, or radiolabels, or determined mathematically, such as deconvolution, upon its plasma concentration profiles.

The term “AUC” (i.e., “area under the curve,” “area under the concentration curve,” or “area under the concentration-time curve”) is a pharmacokinetic term used to refer a method of measurement of bioavailability or extent of absorption of a drug based on a plot of an individual or pool of individual's blood plasma concentrations sampled at frequent intervals; the AUC is directly proportional to the total amount of unaltered drug in the patient's blood plasma. For example, a linear curve for a plot of the AUC versus dose (i.e., straight ascending line) indicates that the drug is being released slowly into the blood stream and is providing a steady amount of drug to the patient; if the AUC versus dose is a linear relationship this generally represents optimal delivery of the drug into the patient's blood stream. By contrast, a non-linear AUC versus dose curve indicates rapid release of drug such that some of the drug is not absorbed, or the drug is metabolized before entering the blood stream.

The term “Cmax” (i.e., “maximum concentration”) is a pharmacokinetic term used to indicate the peak concentration of a particular drug in the blood plasma of a patient. The term “Cmin” (i.e., “minimum concentration”) is a pharmacokinetic term used to indicate the minimum concentration of a particular drug in the blood plasma of a patient.

The term “Tmax” (i.e., “time of maximum concentration” or “time of Cmax”) is a pharmacokinetic term used to indicate the time at which the Cmax is observed during the time course of a drug administration. As would be expected, a dosage form that would include an immediate release as well as a gastric retentive component would have a Tmax that is higher than the Tmax for an immediate release dosage form, but lower than the Tmax for a purely gastric retentive dosage form.

“Preventing,” in reference to a disorder or unwanted physiological event in a patient, refers specifically to inhibiting or reducing the occurrence of symptoms associated with the disorder and/or the underlying cause of the symptoms.

“Therapeutically effective amount,” in reference to a therapeutic agent, refers to an amount that is effective to achieve a desired therapeutic result. Therapeutically effective amounts of a given agent will typically vary with respect to factors such as the type and severity of the disorder or disease being treated and the age, gender, weight and other factors of the patient.

“Treating,” “treat,” and “treatment” refer to reduction in severity and/or frequency of symptoms, elimination of symptoms and/or underlying cause, prevention of the occurrence of symptoms and/or their underlying cause, and improvement or remediation of damage.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the present disclosure or its claims.

### II. GASTRIC RETENTIVE DOSAGE FORM FOR THE EXTENDED RELEASE OF PHENYLEPHRINE

The pharmaceutical compositions described herein, i.e., gastric retained dosage forms comprising phenylephrine, provide both immediate release and extended or sustained release of phenylephrine to the upper gastrointestinal tract. The presently described dosage forms provide for extended release of phenylephrine in the stomach wherein the dosage forms are comprised of a polymer matrix that swells upon imbibition of fluid to a size sufficient for gastric retention. Thus, in formulating the dosage forms, properties which simultaneously allow: a) an extent of swelling to provide gastric retention over an extended period, and b) a rate of swelling and erosion that allows release of the phenylephrine over a time period of approximately 8 hours to about 11 hours or about 9 to about 10 hours, are preferably provided.

The formulation of these pharmaceutical oral dosage forms preferably result in final products that meet the requirements of regulatory agencies such as the Food and Drug Administration. For example, final dosage forms are preferably stable such that they do not fracture during storage and transport. This is measured for tablets, in part, in terms of friability and hardness. Dosage forms preferably also meet requirements for content uniformity, such that dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The dosage form as described here is capable of swelling dimensionally unrestrained in the stomach upon contact with gastric fluid due to the hydrophilic polymer(s) component, such as, polyethylene oxide and/or hypromellose (also known as hydroxypropyl methylcellulose or HPMC), in the formulation, and increase to a size sufficient to be retained in the stomach in a fed mode.

The water-swellable polymer forming the matrix in accordance with this dosage forms described herein is any polymer that is non-toxic, that swells in a dimensionally unrestricted manner upon imbibition of water, and that provides for sustained release of an incorporated drug. Examples of polymers suitable for use in this invention are cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly(2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

The terms “cellulose” and “cellulosic” are used herein to denote a linear polymer of anhydroglucose. Preferred cellulosic polymers are alkyl-substituted cellulosic polymers that ultimately dissolve in the gastrointestinal (GI) tract in a predictably delayed manner. Preferred alkyl-substituted cellulose derivatives are those substituted with alkyl groups of 1 to 3 carbon atoms each. Examples are methylcellulose, hydroxymethyl-cellulose, hydroxyethylcellulose, hydroxypropylcellulose, hydroxypropylmethylcellulose, and carboxymethylcellulose. In terms of their viscosities, one class of preferred alkyl-substituted celluloses includes those whose viscosity is within the range of about 100 to about 110,000 centipoise as a 2% aqueous solution at 20° C. Another class includes those whose viscosity is within the range of about 1,000 to about 4,000 centipoise as a 1% aqueous solution at 20° C. Particularly preferred alkyl-substituted celluloses are hydroxyethylcellulose and hydroxypropylmethylcellulose. A presently preferred hydroxyethylcellulose is NATRASOL® 250HX NF (National Formulary), available from Aqualon Company, Wilmington, Del., USA.

Water-swellable, erodible polymers suitable for use herein are those that swell in a dimensionally unrestrained manner upon contact with water, and gradually erode over time. Examples of such polymers include polyalkylene oxides, such as polyethylene glycols, particularly high molecular weight polyethylene glycols; cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; chitosan; poly(vinyl alcohol); xanthan gum; maleic anhydride copolymers; poly(vinyl pyrrolidone); starch and starch-based polymers; maltodextrins; poly(2-ethyl-2-oxazoline); poly(ethyleneimine); polyurethane; hydrogels; crosslinked polyacrylic acids; and combinations or blends of any of the foregoing.

Polysaccharide gums, both natural and modified (semi-synthetic) can be used. Examples are dextran, xanthan gum, gellan gum, welan gum and rhamsan gum.

Swellable, erodible hydrophilic polymers suitable for forming the gastric retentive portion of the dosage forms described herein include poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×10⁵ Daltons to about 10×10⁶ Daltons. A preferred molecular weight poly(ethylene oxide) polymer is about 5×10⁶ Daltons and is commercially available from The Dow Chemical Company (Midland, Mich.) referred to as SENTRY® POLYOX® water-soluble resins, NF (National Formulary) grade WSR Coagulant. The viscosity of a 1% water solution of the polymer at 25° C. preferably ranges from 4500 to 7500 centipoise.

Dosage forms prepared for oral administration according to the present disclosure will generally contain other inactive additives (excipients) such as binders, lubricants, disintegrants, fillers, stabilizers, surfactants, coloring agents, and the like.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

The dosage form may contain in the IR layer, the ER layer, or both layers, an anti-oxidant for increased stability of the active ingredient as well as the dosage form as a whole. The anti-oxidant may be selected from ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate.

The dosage form may contain in the IR layer, the ER layer, or both layers, a chelating agent. Chelating agents tend to form complexes with trace amount of heavy metal ions inactivating their catalytic activity in the oxidation of medicaments. Ethylenediamine tetracetic acid (EDTA) and its salts, dihydroxy ethyl glycine, citric acid and tartaric acid are most commonly used chelators.

Lubricants are used to facilitate tablet manufacture, promoting powder flow and preventing particle capping (i.e., particle breakage) when pressure is relieved. Useful lubricants are magnesium stearate (in a concentration of from 0.25 wt % to 3 wt %, preferably 0.2 wt % to 1.0 wt %, more preferably about 0.3 wt %), calcium stearate, stearic acid, and hydrogenated vegetable oil (preferably comprised of hydrogenated and refined triglycerides of stearic and palmitic acids at about 1 wt % to 5 wt %, most preferably less than about 2 wt %). Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

The gastric retentive dosage form may be a single layer, bilayer, or multilayer tablet or it may be a capsule. The tablet comprises a gastric retentive layer comprised of phenylephrine dispersed in a matrix of at least one hydrophilic polymer which swells upon imbibition of fluid.

### III. PHENYLEPHRINE

Phenylephrine, known chemically as (R)-3-[-1-hydroxy-2-(methylamino)ethyl]phenol alcohol hydrochloride, is a synthetic, optically active sympathomimetic amine which has one hydroxyl group on the benzene ring. The hydroxyl group is placed in the position meta to the aliphatic side chain. The meta position affords optimal activity and phenylephrine (neo-synephrine) replaced an older preparation, synephrine, in which the hydroxyl was in the para position. Phenylephrine has an approximate molecular weight of 167 and is highly soluble in water. The term phenylephrine includes, but is not limited to pharmaceutically acceptable salts, esters, isomers or derivatives thereof.

### IV. METHODS FOR MAKING THE DOSAGE FORMS

In one embodiment, manufacture of a pharmaceutical oral dosage form that both delivers a therapeutically effective ingredient over a desired period of time and satisfies criteria for commercial and regulatory approval is provided.

In the case of gastric retentive tablets containing phenylephrine, as disclosed herein, tablets may be made by direct compression or by a granulation procedure. Direct compression is used with a group of ingredients can be blended, placed onto a tablet press, and made into a perfect tablet without any of the ingredients having to be changed. Powders that can be blended and compressed are commonly referred to as directly compressible or as direct-blend formulations. When powders do not compress correctly, they must be granulated.

Granulation is a manufacturing process that increases the size and homogeneity of active pharmaceutical ingredients and excipients in a solid dosage formulation. The granulation process, which is often referred to as agglomeration, changes physical characteristics of the dry formulation, with the aim of improving manufacturability, and therefore, product quality.

Granulation technology can be classified into one of two basic types: wet granulation and dry granulation. Wet granulation is the more prevalent agglomeration process utilized within the pharmaceutical industry. Most wet granulation procedures follow some basic steps; the drug(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. There are four basic types of wet granulation; high shear granulation, fluid bed granulation, extrusion and spheronization and spray drying.

A. Fluid Bed Granulation

The fluid bed granulation process involves the suspension of particulates within an air stream while a granulation solution is sprayed down onto the fluidized bed. During the process, the particles are gradually wetted as they pass through the spay zone, where they become tacky as a result of the moisture and the presence of binder within the spray solution. These wetted particles come into contact with, and adhere to, other wetted particles resulting in the formation of particles.

A fluid bed granulator consists of a product container into which the dry powders are charged, an expansion chamber which sits directly on top of the product container, a spray gun assembly, which protrudes through the expansion chamber and is directed down onto the product bed, and air handling equipment positioned upstream and downstream from the processing chamber.

The fluidized bed is maintained by a downstream blower that creates negative pressure within the product container/expansion chamber by pulling air through the system. Upstream, the air is “pre-conditioned” to target values for humidity, temperature and dew point, while special product retention screens and filters keep the powder within the fluid bed system.

As the air is drawn through the product retention screen it “lifts” the powder out of the product container and into the expansion chamber. Since the diameter of the expansion chamber is greater than that of the product container, the air velocity becomes lower within the expansion chamber. This design allows for a higher velocity of air to fluidize the powder bed causing the material to enter the spray zone where granulation occurs before loosing velocity and falling back down into the product container. This cycle continues throughout the granulation process.

The fluid bed granulation process can be characterized as having three distinct phases; pre-conditioning, granulation and drying. In the initial phase, the process air is pre-conditioned to achieve target values for temperature and humidity, while by-passing the product container altogether. Once the optimal conditions are met, the process air is re-directed to flow through the product container, and the process air volume is adjusted to a level that will maintain sufficient fluidization of the powder bed. This pre-conditioning phase completes when the product bed temperature is within the target range specified for the process.

In the next phase of the process, the spraying of the granulating solution begins. The spray rate is set to a fall within a pre-determined range, and the process continues until all of the solution has been sprayed into the batch. It is in this phase where the actual granulation, or agglomeration, takes place.

Once the binder solution is exhausted, the product continues to be fluidized with warm process air until the desired end-point for moisture content is reached. This end-point often correlates well with product bed temperature, therefore in a manufacturing environment, the process can usually be terminated once the target product bed temperature is reached. A typical fluid bed process may require only about thirty to forty-five minutes for the granulation step, plus ten to fifteen minutes on either side for pre-conditioning and drying.

As with any of the wet granulation processes, one variable is the amount of moisture required to achieve successful agglomeration. The fluid bed granulation process requires a “thermodynamic” balance between process air temperature, process air humidity, process air volume and granulation spray rate. While higher process air temperature and process air volume add more heat to the system and remove moisture, more granulating solution and a higher solution spray rate add moisture and remove heat via evaporative cooling. These are the process parameters which must be evaluated as a manufacturing process is developed, and the key is understanding the interdependency of each variable.

Additional factors affecting the outcome of the fluid bed granulation process are the amount and type of binder solution, and the method by which the binder is incorporated within the granulation. Other process variables are the total amount of moisture added through the process, and the rate at which the moisture content is increased. These parameters can have an effect on the quality and the characteristics of the granulation. For instance, a wetter fluid bed granulation process tends to result in a stronger granule with a higher bulk density. However, an overly aggressive process, where moisture is added too rapidly, can loose control over achieving the final particle size and particle size distribution objectives.

B. High Shear Granulation

Many pharmaceutical products manufactured by wet granulation utilize a high shear process, where blending and wet massing are accomplished by the mechanical energy generated by an impeller and a chopper. Mixing, densification and agglomeration are achieved through the “shear” forces exerted by the impeller; hence the process is referred to as high shear granulation.

The process begins by adding the dry powders of the formulation to the high shear granulator, which is a sealed “mixing bowl” with an impellor which rotates through the powder bed, and a chopper blade which breaks up over-agglomerates which can form during the process. There are typically three phases to the high shear process; dry mixing, solution addition, or wet massing and high shear granulation.

In the first phase, dry powders are mixed together by the impeller blade which rotates through the powder bed. The impeller blade is positioned just off the bottom of the product container. There is a similar tolerance between the tips of the impeller blade and the sides of the container. The impeller blades rotation trough the powder bed creates a “roping” vortex of powder movement. The dry mixing phase typically lasts for only a few minutes.

In the second phase of the process, a granulating liquid is added to the sealed product container, usually by use of a peristaltic pump. The solution most often contains a binder with sufficient viscosity to cause the wet massed particles to stick together or agglomerate. It is common for the solution addition phase to last over a period of from three to five minutes. While the impeller is rotating rather slowly during this step of the process, the chopper blade is turning at a fairly high rate of speed, and is positioned within the product container to chop up over-sized agglomerates, while not interfering with the impellers movement.

Once the binder solution has been added to the product container, the final stage of the granulation process begins. In this phase, high shear forces are generated as the impeller blades push through the wet massed powder bed, further distributing the binder and intimately mixing the ingredients contained therein. The impeller and chopper tool continue to rotate until the process is discontinued when the desired granule particle size and density end-points are reached. This end-point is often determined by the power consumption and/or torque on the impeller.

Once the high shear granulation process has been completed, the material is transferred to a fluid bed dryer, or alternatively, spread out onto trays that are then placed in a drying oven, where the product is dried until the desired moisture content is achieved, usually on the order of 1-2% as measured by Loss On Drying (LOD) technique.

A variable that affects the high shear process is the amount of moisture required to achieve a successful granulation. A key to the process is having the right amount of moisture to allow for agglomeration to occur. Too little moisture will result in an under-granulated batch, with weak bonds between particles and smaller, to non-existent particles, with properties similar to those of the dry powder starting materials. On the other hand, excess moisture can result in a “crashed” batch with results varying from severe over-agglomeration to a batch that appears more like soup.

Other formulation parameters affecting the outcome of the high shear granulation process are the amount and type of binder solution, and the method by which the binder is incorporated within the granulation. For example, it is possible to include some of the binder in the dry powder mixture as well as in the granulating solution, or it may be incorporated only in the granulating solution or only in the dry powder, as is the case where water is used as the granulating solution.

The high shear granulation process parameters which are variable include impeller and chopper speeds, the solution addition rate, and the amount of time allocated to the various phases of the process. Of these, preferred variables for consideration are the solution addition rate and the amount of time the wet massed product is under high shear mixing.

C. Extrusion and Spheronization

This specialized wet granulation technique involves multiple processing steps and was developed to produce very uniform, spherical particles ideally suited for multi-particulate drug delivery of delayed and sustained release dosage forms.

Similar to high shear granulation initially, the first step involves the mixing and wet massing of the formulation. Once this step is complete, the wet particles are transferred to an extruder that generates high forces used to press the material out through small holes in the extruder head. The extrudate is of uniform diameter and is then transferred onto a rotating plate for spheronization. The forces generated by the rotating plate initially break up the extruded formulation strands into uniform lengths. Additional dwell time within the spheronizer creates particles that are round and uniform in size. These pellets or spheres are then dried to the target moisture content, usually within a fluid bed system.

Particles produced in this manner tend to be dense, and have a capacity for high drug loading, approaching 90% or more in some cases. The particle size is uniform, and the size distribution is narrow, as compared to other granulation approaches. This quality assures consistent surface area within and between batches, which is desired when functional coatings are subsequently applied to create sustained release formulations, delayed release formulations and formulations designed to target a specific area within the body.

Uniform surface area is desired because the pharmaceutical coating process endpoint is determined not by coating thickness, but by the theoretical batch weight gain of the coating material. If the batch surface area is consistent, then the coating thickness will also be consistent for a given weight gain, and coating thickness is the primary variable in determining the functionality of the coating system, whether the goal is controlling the duration of sustained release formulations or imparting an acid resistant characteristic to “beads” necessary to protect certain compounds which would otherwise be severely degraded in the presence of the acidic environment of the stomach.

D. Spray Drying

Spray drying is a unique and specialized process that converts liquids into dry powders. The process involves the spraying of very finely atomized droplets of solution into a “bed” or stream of hot process air or other suitable gas. Not typically utilized for the conventional granulation of dosage form intermediates, spray drying has gained acceptance within the industry as a robust process that can improve drug solubility and bioavailability.

Spray drying can be used to create co-precipitates of a drug/carrier that can have improved dissolution and solubility characteristics. In addition, the process can also be useful as a processing aid. For example, it is much more difficult to maintain the uniformity of a drug in suspension, as compared to the same compound in solution. One may have a need to develop an aqueous coating or drug layering process utilizing a drug that is otherwise not soluble in water. By creating a co-precipitate of the drug and a suitable water soluble carrier, often a low molecular weight polymer, the co-precipitate will remain in solution throughout the manufacturing process, improving uniformity of the spray solution and the dosage form created by the coating process. Uniformity is particularly desired where lower doses of potent compounds are intended to be coated onto beads or tablet cores.

This same process may be used to enhance the solubility and bioavailability of poorly soluble drugs. By complexing certain excipients and the active ingredient within a solvent system which is then spray dried, it is possible to enhance the drugs absorption within the body. Selection of the solvent system, the complexing agent(s) and the ratios utilized within the formulation are formulation variables that influence the effectiveness of solubility enhancement utilizing the spray drying technique. Other process parameters with an effect on drug solubility are the temperatures of the spray solution and process gas, the spray rate and droplet size and the rate of re-crystallization. The spray dried granulations created by these techniques can then be incorporated into capsules or tablets by conventional manufacturing processes.

E. Dry Granulation

The dry granulation process involves three basic steps; the drug(s) and excipients(s) are mixed (along with a suitable binder if needed) and some form of lubrication, the powder mixture is compressed into dry “compacts,” and then the compacts are sized by a milling step. The two methods by which dry granulation can be accomplished are slugging and roller compaction.

### IV. METHODS OF MAKING THE EXTENDED RELEASE GASTRIC RETENTIVE DOSAGE FORMS DISCLOSED HEREIN

In one aspect, a method of making a gastric retentive extended-release dosage form as a single layer tablet comprising dry blending of the phenylephrine with water-swellable polymers and other excipients is provided. The dry blended mixture is then compressed into tablets.

Extended release polymer matrices comprising phenylephrine can be made using either POLYOX® 1105 (approximate molecular weight of 900,000 Daltons), POLYOX® N-60K (approximate molecular weight of 2,000,000 Daltons), or POLYOX® WSR-301 (approximate molecular weight of 4,000,000 Daltons). Alternatively, the matrix comprises a cellulose-derived polymer.

Bulk and tap densities for the tablets can be determined as follows. A graduated cylinder is filled with a certain amount of material, and the volume recorded to determine the material bulk density. Tap density can be determined with a help of a Tap Density Tester by exposing the material to 100 taps per test and recording the new volume.

Particle size determination is performed immediately after granulation, after sieving through 20 mesh screen to remove agglomerates. Particle diameter is determined with a sieve-type particle diameter distribution gauge using sieves with openings of 44, 53, 75, 106, 150, and 250 mesh. Fractions are weighed on Mettler balance to estimate size distribution. This provides determination of the quantitative ratio by particle diameter of composition comprising extended release particles. Sieve analysis according to standard United States Pharmacopoeia methods (e.g., USP-23 NF 18), may be done such as by using a Meinzer II Sieve Shaker.

The granulated mixture can be blended with the polymer, filler and lubricant in a V-blender. The resultant mixture can be compressed into monolithic, single-layer tablets using a Manesty® BB4 press, with a modified oval 0.3937″ width×0.6299″ length×0.075″ cup depth tool. Tablets may be prepared at a rate, for example, of approximately 800 tablets per minute.

Tablets can then be characterized for disintegration and dissolution release profiles, hardness, friability and content uniformity.

The dissolution profiles for the tablets are determined in USP apparatus (40 mesh baskets), 100 revolutions per minute (rpm), in pH 5.8 phosphate buffer (0.1 N HCl) or in modified simulated gastric fluid, 37° C. Samples of 5 milliliters (ml) at each time-point, are taken without media replacement at 1, 2, 4, 6, 8 and 12 hours. The resulting cumulative dissolution profiles for the tablets are based upon a theoretical percent active added to the formulations.

A disintegration tester measures the time it takes a tablet to break apart in solution. The tester suspends tablets in a solution bath for visual monitoring of the disintegration rate. Both the time to disintegration and the disintegration consistency of all tablets are measured. The disintegration profile is determined in a USP Disintegration Tester in pH 5.8 phosphate buffer. Samples, 1 ml at each time-point, may be taken, for example, without media replacement at 0.5, 1, 2, 3, 4, 5, 6, 7 and 8 hours. The resulting cumulative disintegration profiles are based upon a theoretical percent active added to the formulation is determined.

Tablet hardness changes rapidly after compression as the tablet cools. In the case of the presently disclosed gastric retentive dosage forms, a tablet that is too hard may not be able to imbibe fluid rapidly enough to prevent passage through the pylorus in a stomach in a fed mode. A tablet that is too soft may break apart, not handle well, and can create other defects in manufacturing. A soft tablet may not package well or may not stay together in transit.

After tablets are formed by compression, it is desired that the tablets have a strength of at least 9-25 Kiloponds (Kp)/cm², preferably at least about 12-20 (Kp)/cm². A hardness tester is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially relevant during any transport of the dosage form as any fracturing of the final dosage form will result in a subject receiving less than the prescribed medication. Friability can be determined using a Roche Friability Drum according to standard USP guidelines that specify the number of samples, the total number of drum revolutions and the drum rpm to be used. Friability values of from 0.8 to 1.0% are regarded as constituting the upper limit of acceptability.

The prepared tablets are tested for content uniformity to determine if they meet the pharmaceutical requirement of <6% relative standard deviation (RSD). Each tablet is placed in a solution of 1.0 N HCl and stirred at room temperature until all fragments have visibly dissolved. The solution containing the dissolved tablet is analyzed by HPLC.

In another aspect, a method of making a bilayer tablet comprising a gastric retentive extended-release layer and an immediate release layer is provided.

### V. STABILITY OF PHENYLEPHRINE EXTENDED RELEASE FORMULATIONS

Stability testing is the primary tool used to assess expiration dating and storage conditions for pharmaceutical products. Many protocols have been used for stability testing, but most in the industry are now standardizing on the recommendations of the International Conference on Harmonization (ICH). These guidelines were developed as a cooperative effort between regulatory agencies and industry officials from Europe, Japan, and the United States.

Stability testing includes long-term studies, where the product is stored at room temperature and humidity conditions, as well as accelerated studies where the product is stored under conditions of high heat and humidity. Proper design, implementation, monitoring and evaluation of the studies are crucial for obtaining useful and accurate stability data. Stability studies are linked to the establishment and assurance of safety, quality and efficacy of the drug product from early phase development through the lifecycle of the drug product. Stability data for the drug substance are used to determine optimal storage and packaging conditions for bulk lots of the material. The stability studies for the drug product are designed to determine the expiration date (or shelf life). In order to assess stability, the appropriate physical, chemical, biological and microbiological testing must be performed. Usually this testing is a subset of the release testing.

Studies are designed to degrade the solid drug substance and appropriate solutions, allowing the determination of the degradation profile. The drug substance is usually challenged under a variety of accelerated environmental conditions to evaluate its intrinsic stability and degradation profile.

HPLC is the predominant tool used to analyze the drug substance and the impurities, particularly for small molecules. Frequently, the same HPLC method may be used for drug substance and drug product, although different sample preparation methods would normally be required. Often the assay and impurity testing can be performed using a single HPLC method. However, the assay and purity determinations may also be separate methods. At least in the U.S., full validation of the analytical method is not required until the end of Phase 2 clinical trials, but the establishment of specificity, linearity and limit of quantification (for impurities) is considered at the earliest stages, since verification of stability hinges on a suitable method for separating impurities from the active ingredient and at least quantifying the impurities relative to the drug substance.

Stress studies at elevated temperature (e.g., 50° C., 60° C. and 70° C.) for several weeks may be performed to assess thermal stability. Provided the degradation mechanism is the same at the different temperatures used, kinetic or statistical models can be used to determine the rate of degradation at other temperatures (e.g., 25° C.). The solid stability should also be performed in the presence and absence of water vapor to assess the dependence of stability on humidity.

Degradation studies should also be performed in solution. The solvent used for the solution testing will depend on the solubility of the drug substance and should include water, if the drug substance is water-soluble. Other solutions or solvent systems may be evaluated depending on the anticipated formulation or the synthetic process. A series of buffered solutions in the pH range 2-9 are useful in assessing the impact of solution pH on the degradation. Photostability should also be evaluated. A xenon light source can be used as a stress condition. Alternatively, one can use an accelerated version of either Options 1 or 2 as described in the ICH guideline for determination of photostability. Oxidation of the drug substance under accelerated conditions (e.g., hydrogen peroxide), may also be performed to establish oxidation products that could be formed and sensitivity to oxidative attack.

Early drug product stability studies are designed to help establish a suitable formulation for delivery of the drug substance. Compatibility studies of the drug substance with excipients should be performed to eliminate excipients that are not compatible with the drug substance.

### VI. METHODS FOR TREATING CONGESTION

In another aspect, a subject suffering from symptoms including, but not limited to, ophthalmic disorders (hyperaemia of conjunctiva, posterior synechiae, acute atopic), nasal congestion, hemorrhoids, hypotension, shock, hypotension during spinal anesthesia, and paroxysmal supraventricular tachycardia, may be treated with the herein described dosage forms. Moreover, phenylephrine may also be used as an aid in the diagnosis of heart murmurs and for prolongation of spinal anesthesia.

Generally, the frequency of administration of a particular dosage form is determined to provide the most effective results in an efficient manner without overdosing and varies according to the following criteria: (1) the characteristics of the particular drug(s), including both its pharmacological characteristics and its physical characteristics, such as solubility; (2) the characteristics of the swellable matrix, such as its permeability; and (3) the relative amounts of the drug and polymer. In most cases, the dosage form is prepared such that effective results are achieved with administration once every eight hours, once every twelve hours, or once every twenty-four hours. As previously discussed, due to the physical constraints placed on a tablet or capsule that is to be swallowed by a patient, most dosage forms can only support a limited amount of drug within a single dosage unit.

In one embodiment, the dosage form allows a dosing frequency of two times a day (b.i.d.) or three times a day (t.i.d.) to result in sustained plasma concentration of the active ingredient as compared to current immediate release products that require more frequent administration for effective therapy.

Within the context of the present disclosure, the gastric retentive dosage forms have the advantage of improving patient compliance with administration protocols because the drugs may be administered in a once-daily or twice-daily dosing regimen, rather than the multiple dosing administrations necessary for the immediate release dosage forms of phenylephrine in order to maintain a desired level of relief. One embodiment of the invention relates to a method of administering a therapeutically effective amount of phenylephrine to a patient in need thereof, comprising administering the phenylephrine or pharmaceutically acceptable salts thereof, in a gastric retentive dosage form once in the morning or evening in a once a day daily regime. Another embodiment comprises administering the gastric retentive dosage form twice a day, for example once in the morning and once in the evening in a twice a day daily dosage regime.

For all modes of administration, the gastric retentive dosage forms described herein are preferably administered in the fed mode, i.e., with or just after consumption of a small meal (see U.S. Publication No. 2003/0104062, herein incorporated by reference). When administered in the evening fed mode, the gastric retentive dosage form may provide the subject with continued relief from congestion through the night and into the next day. The gastric retentive dosage form of the present invention is able to provide congestive relief for an extended period of time because the dosage form allows for both extended release of the phenylephrine and the superior absorption of the drug in the GI tract.

In some aspects, the postprandial or fed mode can also be induced pharmacologically, by the administration of pharmacological agents that have an effect that is the same or similar to that of a meal. These fed-mode inducing agents may be administered separately or they may be included in the dosage form as an ingredient dispersed in the shell, in both the shell and the core, or in an outer immediate release coating. Examples of pharmacological fed-mode inducing agents are disclosed in U.S. Pat. No. 7,405,238, entitled “Pharmacological Inducement of the Fed Mode for Enhanced Drug Administration to the Stomach,” inventors Markey, Shell, and Berner, the contents of which are incorporated herein by reference.

The ability to treat a subject though a once- or twice-daily dosing schedule has a distinct advantage over the more common thrice-daily dosing of current marketed forms of extended release phenylephrine. This advantage involves both convenience and more stable drug levels in the blood. This once- or twice-daily dosage form requires that the dosage form contains enough phenylephrine to provide therapeutic efficacy over an extended period approximating twelve hours.

### EXAMPLES

The following examples illustrate certain aspects and advantages of the present invention, however, the present invention is in no way considered to be limited to the particular embodiments described below.

### Example 1

### Formulation of Prototype I Gastric Retentive Tablets Having 30 Mg Phenylephrine

Prototype gastric retentive (GR) tablets containing 30 mg phenylephrine in a gastric retentive layer were developed.

Gastric retentive phenylephrine (PE) tablets were manufactured using a dry blend process, and hand made on a Carver Auto C Press (Fred Carver, Inc., Indiana). The dry blend process consisted of blending all the ingredients in a glass jar, and compressing into a tablet (30 mg PE dose) using different size and shape die (Natoli Engineering, St. Charles, Mo.). The parameters for the operation of the Carver Auto C Press were as follows: 3000 lbs force, 0 second dwell time (the setting on the Carver Press), and 100% pump speed.

The formulations for three tablet prototypes (“GR Prototype I-6 Hr Release”) designed for 6 hour release are set forth in Table 1 below.

TABLE 1

| | Formula A | | Formula B | | Formula C | |
|---|---|---|---|---|---|---|
| Ingredient | Mg | wt % | Mg | wt % | Mg | wt % |
| Phenylephrine HCl | 30 | 5.0 | 30.0 | 10.0 | 30.0 | 5.0 |
| POLYOX ® 301 | 180.0 | 30.0 | 180.0 | 60.0 | — | — |
| POLYOX ® N60K | — | — | — | — | 180.0 | 30.0 |
| Lactose 316 Fast-Flo | 384.0 | 64.0 | 87.0 | 29.0 | 384.0 | 64.0 |
| Magnesium Stearate | 6.0 | 1.0 | 3.0 | 1.0 | 6.0 | 1.0 |
| Total | 600.0 | 100.0 | 300.0 | 100.0 | 600.0 | 100.0 |

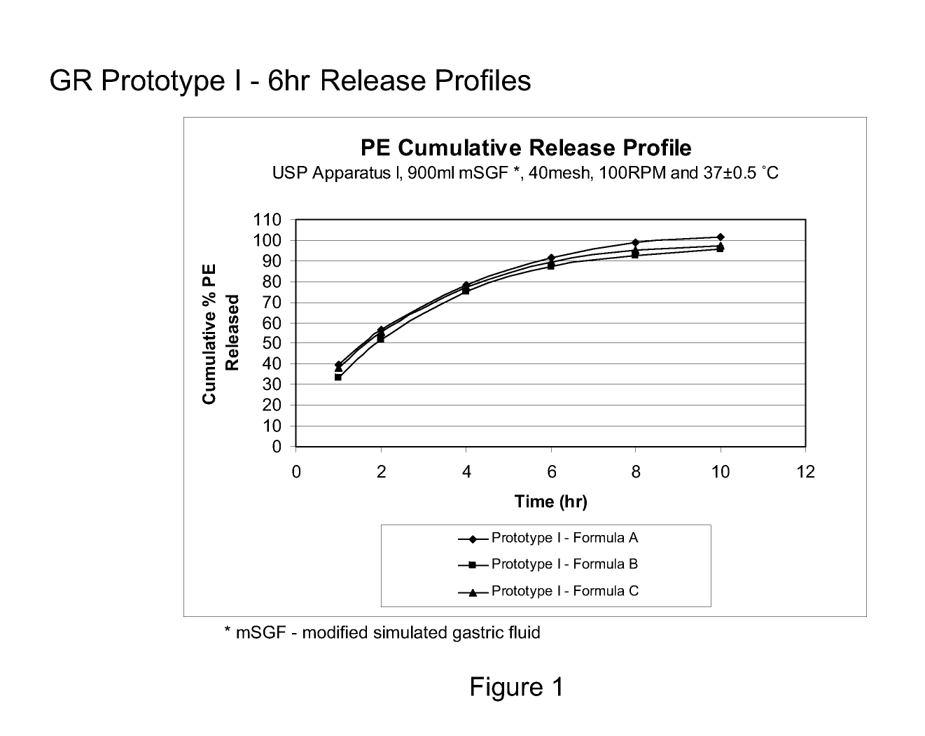

Release rate characteristics of the GR Prototype I-6 Hr Release tablets were investigated using a USP Dissolution Apparatus 1 containing 900 mg modified simulated gastric fluid, a 40 mesh basket, at 100 rpm and 37±0.5° C. The results are presented in Table 2 below and graphically shown in FIG. 1. FIG. 1 shows that each of the three formulations provide an extended release of the phenylephrine over a time period of about 10 hours.

TABLE 2

| | Cumulative | Cumulative | Cumulative |
|---|---|---|---|
| Time | Release (%) | Release (%) | Release (%) |
| (hours) | Formula A | Formula B | Formula C |
| 1 | 39.9 | 33.5 | 38.1 |
| 2 | 56.4 | 51.8 | 55.3 |
| 4 | 78.5 | 75.2 | 77.4 |
| 6 | 91.7 | 87 | 89.2 |
| 8 | 99.1 | 92.7 | 95.1 |
| 10 | 101.3 | 95.7 | 97.5 |

### Example 2

### Formulation of Prototype II Gastric Retentive Tablets Having 30 Mg Phenylephrine

Tablets referred to herein as GR Prototype II tablets, were made using the methods described above. Two formulas, D and E are listed below in Table 3. Tablets were manufactured using the higher molecular weight POLYOX® 303 (7,000,000 Da) and Advantose® 100 maltose powder (SPI Pharma, Wilmington, Del.).

TABLE 3
GR Prototype II-10 hr Formula

| | Formula D | | Formula E | |
|---|---|---|---|---|
| Ingredient | Mg | wt % | Mg | wt % |
| Phenylephrine HCl | 30 | 5.0 | 30.0 | 7.5 |
| POLYOX ® 303 | 360.0 | 60.0 | 326.0 | 81.5 |
| Advantose ™ 100 | 204.0 | 34.0 | 40.0 | 10.0 |
| Magnesium Stearate | 6.0 | 1.0 | 4.0 | 1.0 |
| Total | 600.0 | 100.0 | 400.0 | 100.0 |

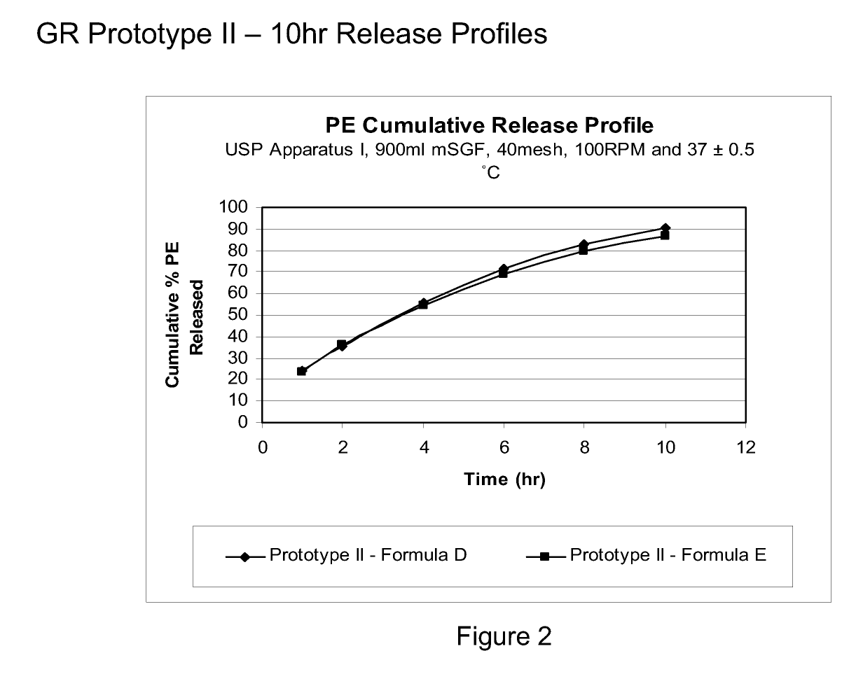

Release rate characteristics of the GR Prototype II-10 Hr Release tablets were investigated using a USP Dissolution Apparatus 1 containing 900 mg modified simulated gastric fluid, a 40 mesh basket, at 100 rpm and 37±0.5° C. The results are presented in Table 4 below and graphically shown in FIG. 2. As seen in FIG. 2, release of substantially all of the phenylephrine occurred over a time period of at least 10 hours. Additionally, the initial release of phenylephrine from formulas D and E was slower than that seen for formulas A, B, and C. Specifically, about 24% of the phenylephrine was released from the tablets of formulas D and E over the first hour of immersion in fluid, as compared to a release of about 33% to 40% of the phenylephrine from the tablets of formulas A, B and C. Accordingly, it appears that the presence of the maltose (e.g., at a weight percent of 10% to 34%) provided a slower release of the phenylephrine within the first hour of immersion in fluid.

TABLE 4

| | Cumulative | Cumulative |
|---|---|---|
| Time | Release (%) | Release (%) |
| (hours) | Formula A | Formula B |
| 1 | 23.8 | 23.7 |
| 2 | 35.7 | 35.9 |
| 4 | 55.7 | 54.6 |
| 6 | 71.5 | 69.1 |
| 8 | 82.9 | 79.7 |
| 10 | 90.3 | 86.9 |

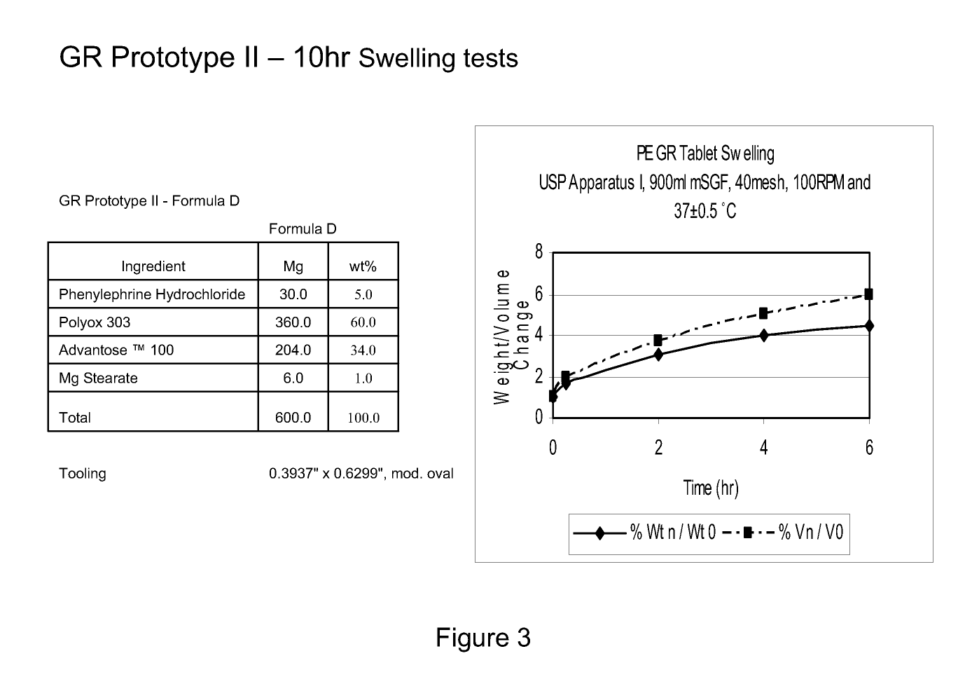

In addition, swelling of the GR Prototype II tablet, Formula D was tested for swelling in simulated gastric fluid. This tablet was pressed with a 0.3937″×0.6299″ mod. oval die. As shown in FIG. 3, the tablets swell to a weight which is about 169% of the original weight within 15 minutes of being immersed in fluid, to a weight which is about 308% of the original weight after 2 hours in fluid, to a weight which is about 402% of the original weight after 4 hours and to a weight which is about 443% of the original weight after 6 hours in simulated gastric fluid. Such swelling properties are sufficient to allow retention of the dosage form within a stomach of the fed mode during release of the drug over a period of several hours.

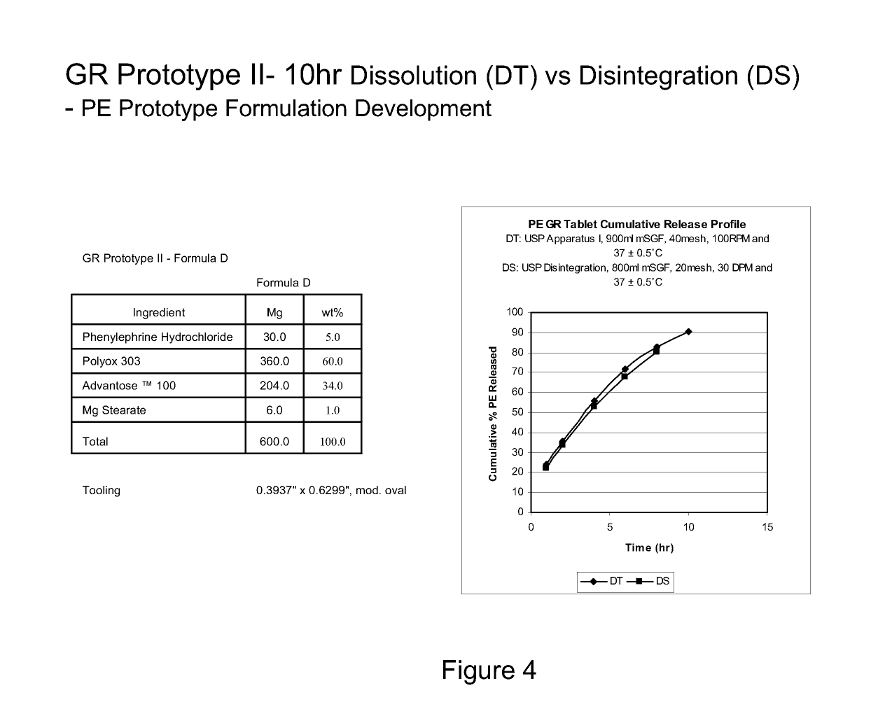

Release rate characteristics of the GR Prototype II, Formula D tablet were observed by measuring both dissolution and disintegration in simulated gastric fluid. The results are presented in Tables 5 below and are presented as a graph in FIG. 4.

TABLE 5
Disintegration and Dissolution of
GR Prototype II, Formula D

| Time | Dissolution (DT) | Disintegration (DS) |
|---|---|---|
| (hours) | % PE released | % PE Released |
| 1 | 23.8 | 22.2 |
| 2 | 35.7 | 33.6 |
| 4 | 55.7 | 53 |
| 6 | 71.5 | 68 |
| 8 | 82.9 | 80.1 |
| 10 | 90.3 | |

### Example 3

### Stability Studies of GR Prototype II Tablets

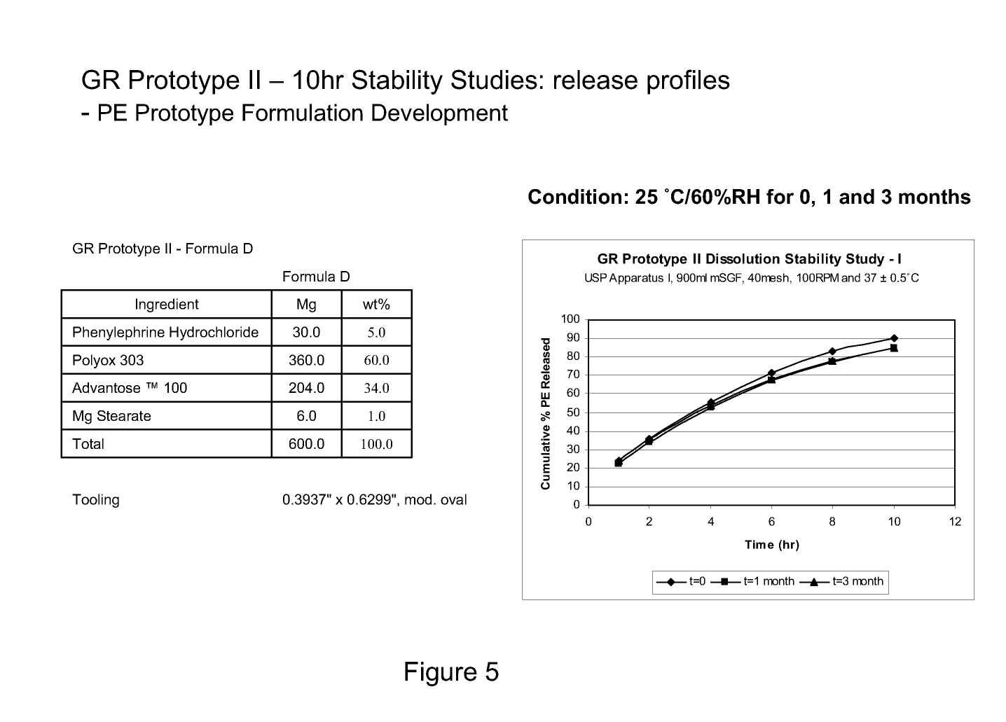

GR Prototype II, Formula D tablets (see Table 3), were stored at 25° C. under conditions of 60% relative humidity (RH) for 0, 1 and 3 months. Dissolution testing was then done to determine the effects of the storage on cumulative phenylephrine release. The results are shown in Table 6 below, and illustrated in FIG. 5. The studies show minimal effect of storage on stability of the phenylephrine dosage form.

TABLE 6
Effects of Storage on PE Release (25° C./60% RH)

| Time | 0 Months | 1 Month | 3 Months |
|---|---|---|---|
| (hours) | % PE released | % PE released | % PE released |
| 1 | 23.8 | 22.3 | 23.8 |
| 2 | 35.7 | 33.8 | 35.4 |
| 4 | 55.7 | 52.5 | 54.0 |
| 6 | 71.5 | 67 | 68.0 |
| 8 | 82.9 | 77.4 | 78.0 |
| 10 | 90.3 | 84.8 | 85.0 |

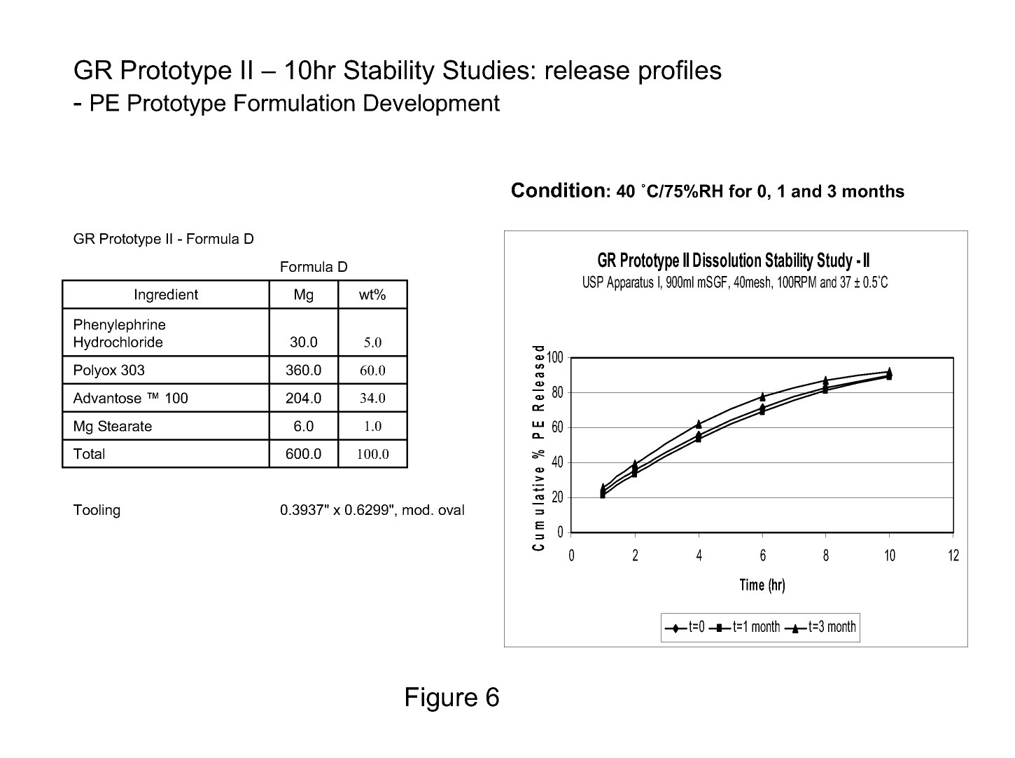

GR Prototype II, Formula D tablets (see Table 3), were also stored at 40° C. under conditions of 75% relative humidity (RH) for 0, 1 and 3 months. Dissolution testing was then done to determine the effects of the storage on cumulative phenylephrine release. The results are shown in Table 7 below, and illustrated in FIG. 6. The studies again show minimal effect of storage on stability of the phenylephrine dosage form with respect to its ability to provide the desired release profile.

The stability of the tablets is also tested with respect to the stability of the phenylephrine itself. HPLC methods as commonly known in the art are used to analyze phenylephrine from the stored dosage form for the presence of impurities or breakdown products.

TABLE 7
Effects of Storage on PE Release (40° C./75% RH)

| Time | 0 Months | 1 Month | 3 Months |
|---|---|---|---|
| (hours) | % PE released | % PE released | % PE released |
| 1 | 23.8 | 21.6 | 25.6 |
| 2 | 35.7 | 33.8 | 39.6 |
| 4 | 55.7 | 53.5 | 62.1 |
| 6 | 71.5 | 69.2 | 77.7 |
| 8 | 82.9 | 81.2 | 87.5 |
| 10 | 90.3 | 89.3 | 92.0 |

The effects of storage were also determined with respect to total phenylephrine content after extended storage. Recovery of phenylephrine from the GR Prototype II, Formula D tablets stored for 0, 1 and 3 months at 25° C./60% RH and 40° C./75% RH are shown below in Table 8.

TABLE 8
Effects of Storage on PE Recovery

| Time | Storage at 25° C./60% RH | Storage at 40° C./75% RH |
|---|---|---|
| (months) | (% Recovery) | (% Recovery) |
| 0 | 99.8 ± 2.12 | 99.8 ± 2.12 |
| 1 | 99.9 ± 1.27 | 96.7 ± 1.33 |
| 3 | 97.6 ± 1.00 | 91.7 ± 2.07 |

### Example 4

### Retention Studies In Vivo

Gastric retention of the GR Prototype II Formula D and Formula E tablets in vivo were studied using dogs. Five dogs were used. Dogs were orally administered the tablet with a meal to induce the fed mode. Each tablet contained two radio-opaque strings in the shape of an “X”. Separation of the strings was considered to signify complete erosion of the tablets. Images were obtained every 30 min until the strings separated. The results showed that the erosion time for the 650 mg GR tablets was 4.6±0.5 hours. This predicts an erosion time of approximately 8 hours in humans. The emptying time for the dogs ranged from 4.25 hours to greater than 6.5 hours, as described in Table 9:

TABLE 9
Erosion Study in Dogs

| Dog | Emptying Time |
|---|---|
| # | (hr) |
| 1 | 4.25 |
| 2 | 6.25 |
| 3 | 4.75 |
| 4 | >6.5 |
| 5 | >6.5 |

The same in vivo studies in which the dogs were administered the GR Prototype II Formulation E tablets showed that all tablets were intact and in the stomach at 6 hours.

### Example 5

### Formulation of GR Bilayer Tablets

A bilayer tablet having both an immediate release (IR) layer containing phenylephrine and a gastric retained extended release (ER) layer containing phenylephrine was produced. The GR bilayer tablets were manufactured using a dry blend process, and hand made on a Carver Auto C Press (Fred Carver, Inc., Indiana). The ER and IR dry blend process consisted of blending all the ingredients in the two glass jars, filling the ER blend in the die first, tapping the ER blend slightly with the up-punch, filling the IR blend in the die and compressing into a tablet (30 mg PE dose) using different size die (Natoli Engineering, St. Charles, Mo.). The parameters for the operation of the carver Auto C Press were as follows: 3000 lbs force, 0 second dwell time (the setting on the Carver Press), and 100% pump speed. The components of the IR and the ER layers are presented in Tables 10 and 11, respectively. Tooling for Formula F tablets was a 0.3937″×0.6299″, mod. oval die. Tooling for Formula G tablets was a 0.3937″×0.7087″ mod. oval die.

TABLE 10
Bilayer Prototype III IR Layer

| | Bi-Layer Prototype I | | Bi-Layer Prototype I | |
|---|---|---|---|---|
| | Formula F | | Formula G | |
| Ingredient | Mg | wt % | Mg | wt % |
| Phenylephrine | 10.0 | 10.0 | 10.0 | 6.7 |
| HCl | | | | |
| Microcrystalline | 88.0 | 88.0 | 137.0 | 91.3 |
| Cellulose, NF | | | | |
| Opadry Blue | 1.0 | 1.0 | 1.5 | 1.0 |
| Magnesium | 1.0 | 1.0 | 1.5 | 1.0 |
| Stearate, NF | | | | |
| Total | 100.0 | 100.0 | 150.0 | 100.0 |

TABLE 11
Bilayer Prototype III ER Layer

| | Bi-Layer Prototype I | | Bi-Layer Prototype I | |
|---|---|---|---|---|
| | Formula F | | Formula G | |
| Ingredient | Mg | wt % | Mg | wt % |
| Phenylephrine | 30.0 | 5.0 | 30.0 | 5.0 |
| HCl | | | | |
| Polyox 303 | 360.0 | 60.0 | 360.0 | 60.0 |
| Microcrystalline | 204.0 | 34.0 | 204.0 | 34.0 |
| Cellulose, NF | | | | |
| Magnesium | 6.0 | 1.0 | 6.0 | 1.0 |
| Stearate | | | | |
| Total | 600.0 | 100.0 | 600.0 | 100.0 |

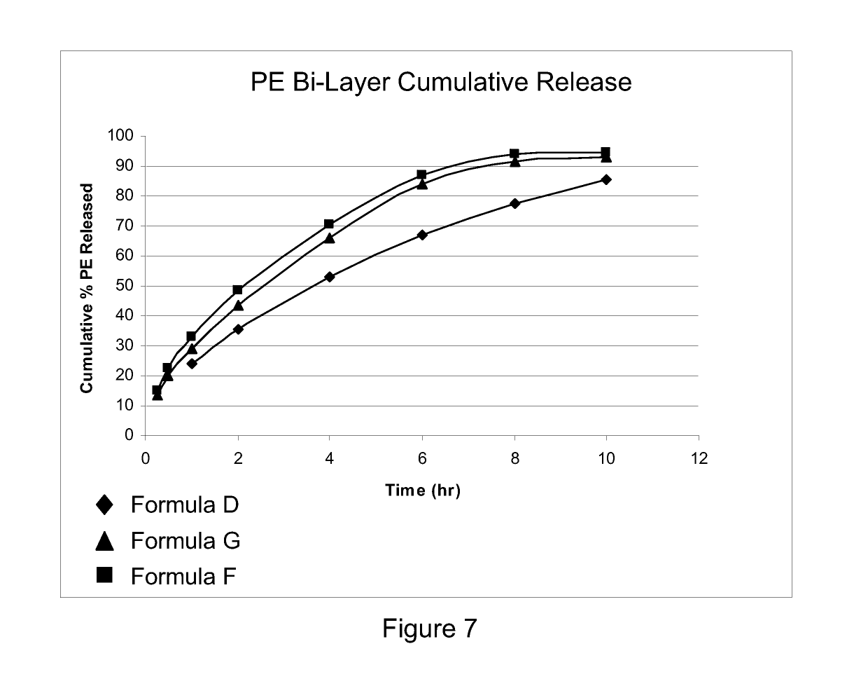

Release rate characteristics of the GR Prototype III bilayer tablets were compared to those of GR prototype II Formula D tablets using a USP Dissolution Apparatus 1 containing 900 mg modified simulated gastric fluid, a 40 mesh basket, at 100 rpm and 37±0.5° C. The results are presented in Table 12 below and graphically shown in FIG. 7.

TABLE 12
Cumulative Release via Dissolution

| Time | GR Prototype II | GR Prototype III | GR Prototype III |
|---|---|---|---|
| (hr) | Formula D | Formula F | Formula F |
| 0.25 | | 15.2 | 13.3 |
| 0.5 | | 22.4 | 19.9 |
| 1 | 24.0 | 32.8 | 29.2 |
| 2 | 35.5 | 48.4 | 43.7 |
| 4 | 53.1 | 70.6 | 65.8 |
| 6 | 66.9 | 87.2 | 83.8 |
| 8 | 77.6 | 94.2 | 91.3 |
| 10 | 85.5 | 94.6 | 92.8 |

### Example 5

### Pharmacokinetic Simulation of Gastric Retentive Phenylephrine HCl Dosage Forms

Pharmacokinetic simulation analysis of phenylephrine for extended-release using gastric retentive dosage forms was performed to predict a one-compartment model with first order absorption and elimination was used. The upper gastrointestinal tract was treated as a “mixing tank,” shown in FIG. 5 below.

### Pharmacokinetic Simulation

Plasma concentration profiles of phenylephrine hydrochloride (20 mg oral solution, fasted administration) were obtained from the literature (Stockis et al., 1995) and were fitted to the pharmacokinetic model below to obtain parameters for calculating the disposition function. Laplace transforms of the input (square-root of time or zero-order drug release from GR systems) and disposition functions were obtained and the product was inverted to obtain plasma concentrations as a function of time. Phenylephrine HCl is freely soluble in water, which ordinarily would suggest a diffusional mechanism (square-root of time) for controlled drug release. However, because of the low loading of the drug in the tablet (20 mg to 30 mg), an erosional system (zero-order release) may be necessary and the actual mechanism may be a combination of both. Simulation of plasma concentrations under multiple dose administration was obtained by superposition of the single doses profiles.

A pharmacokinetic model used for ranitidine oral immediate release is illustrated here:

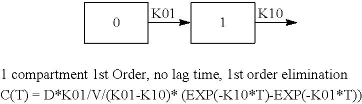

GR Input Parameters (for the Duration of Release)

Square-root of time input rate=Dose/[2×SQRT(Duration of release)]

GRS6: 6-hour duration of square-root of time release

GRS9: 9-hour duration of square-root of time release

Zero-order (constant) drug input rate=Dose/Duration of release

GRZ6: 6-hour duration of zero-order release

GRZ9: 9-hour duration of zero-order release

These durations were chosen assuming phenylephrine absorption is restricted to the upper gastrointestinal (GI) tract.

### I. Results of the Pharmacokinetic Simulation

### A. Single Dose

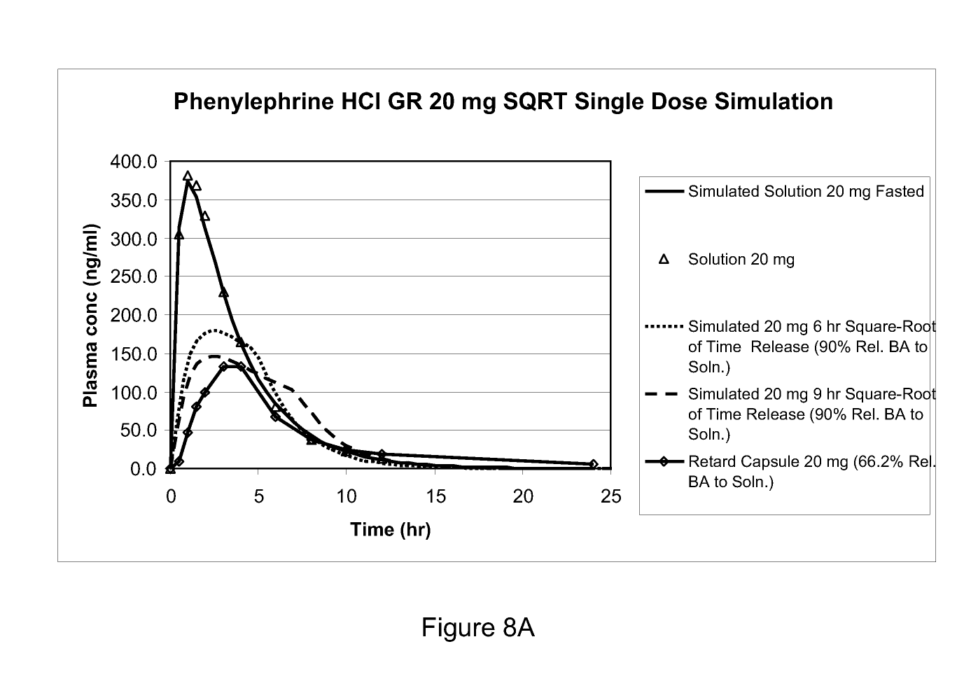

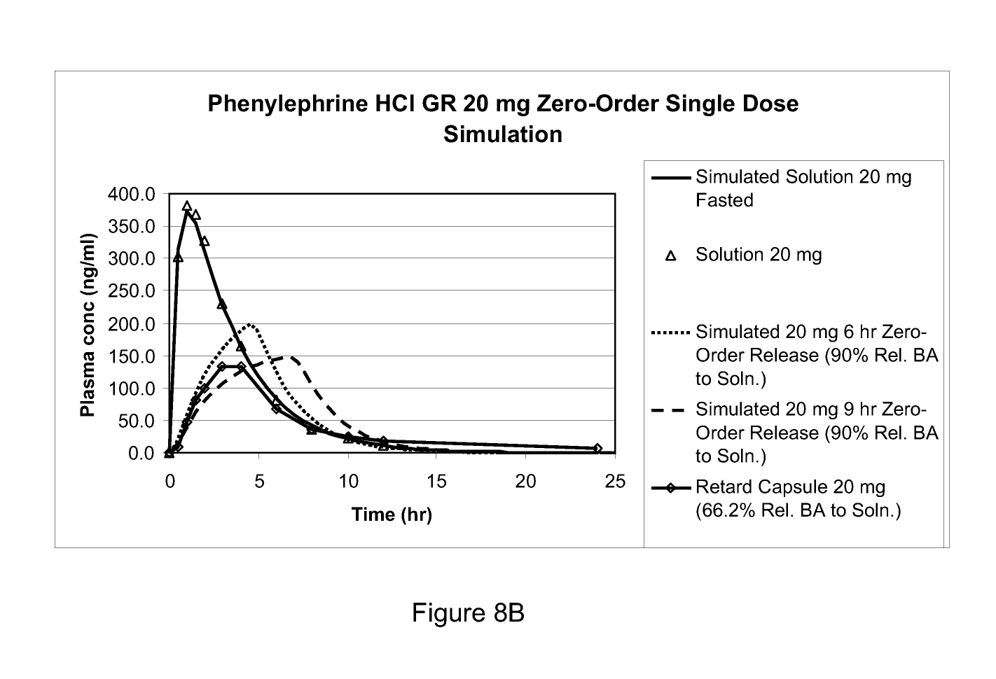

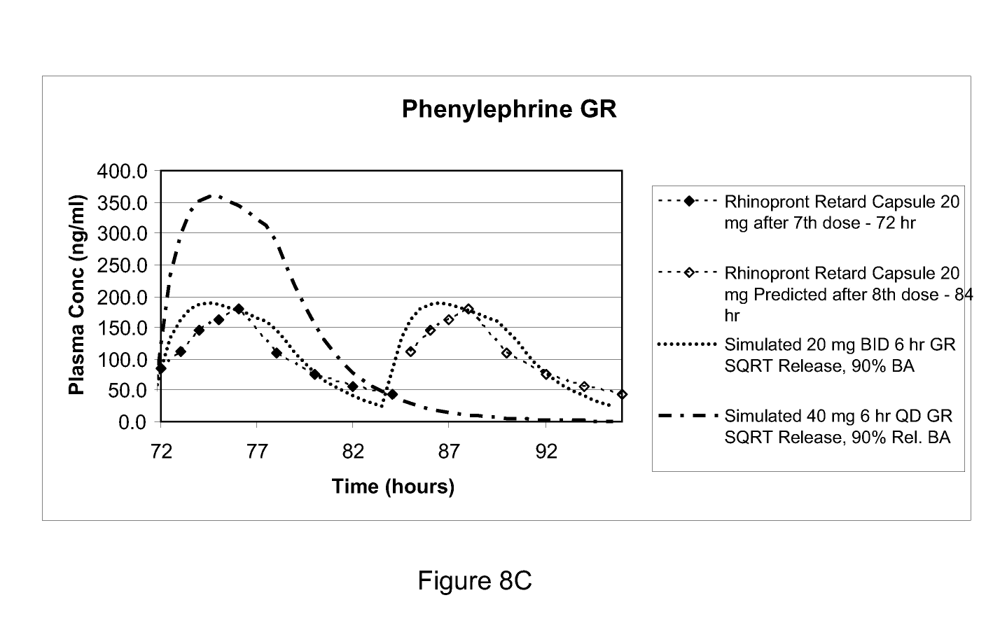

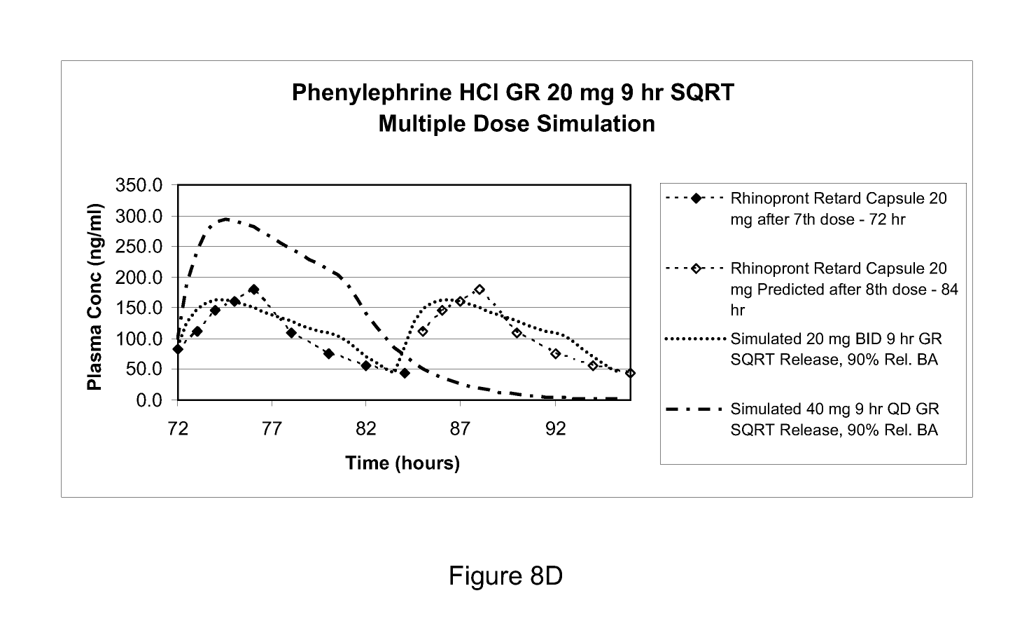

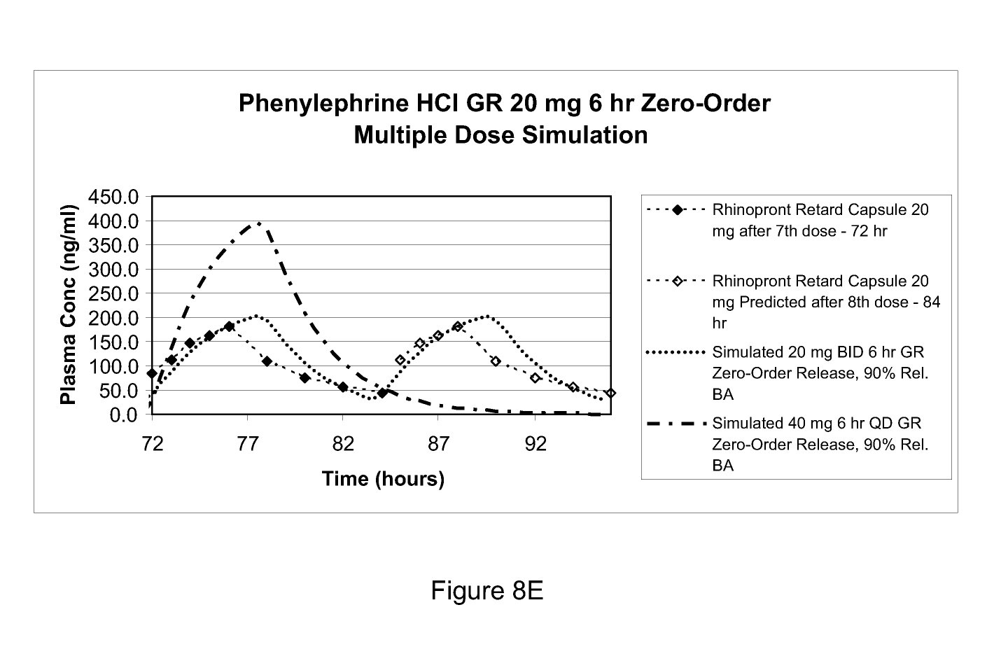

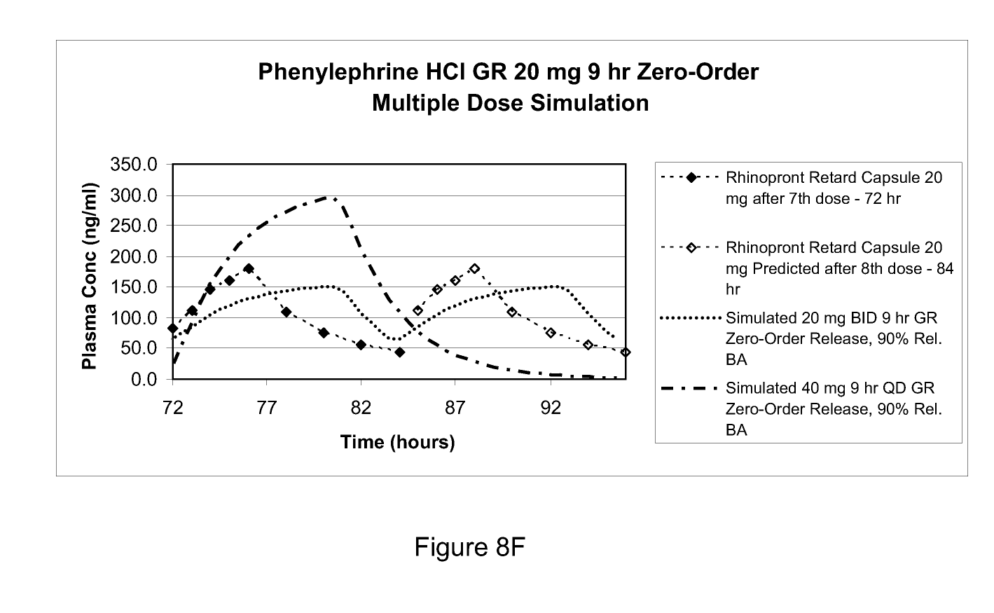

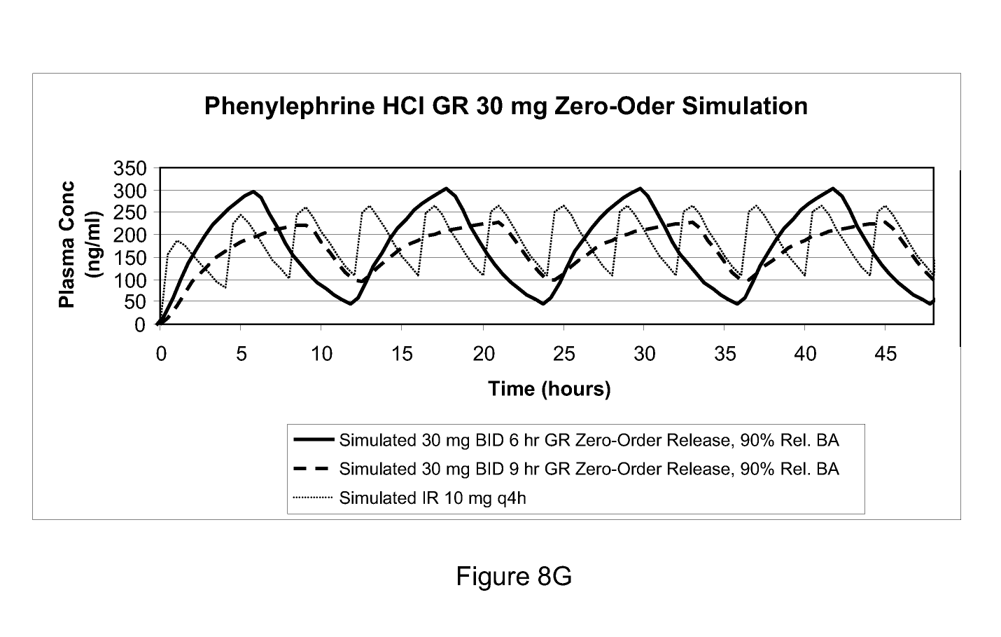

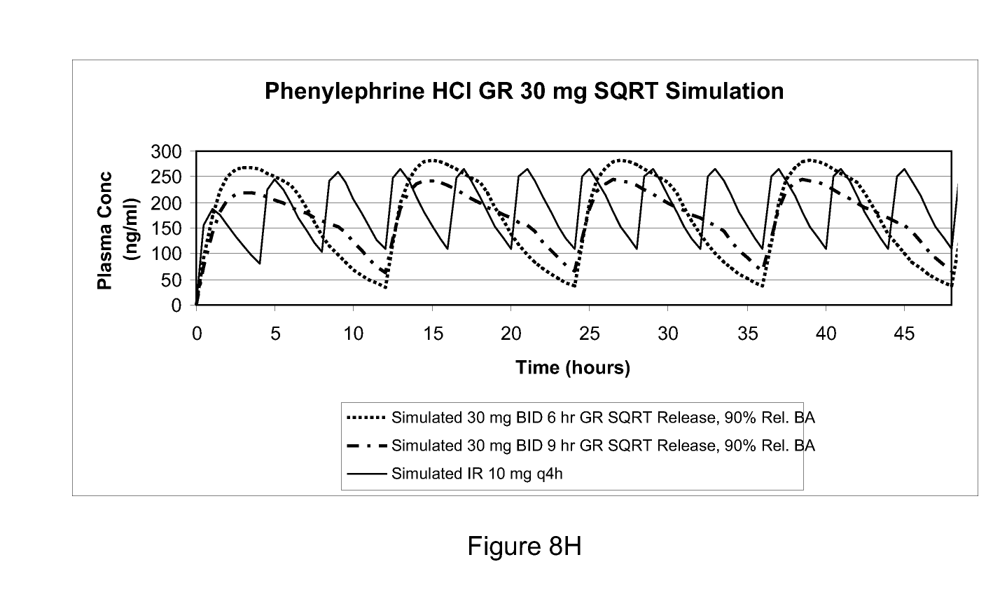

FIG. 8A shows a comparison of simulated plasma concentration profiles provided by single-dose Phenylephrine HCl 20 mg GR systems of 6-hr and 9-hr square-root of time release (GRS6 & GRS9), IR, and Rhinopront® (Stockis et al., 1995).

FIG. 8B shows a comparison of simulated plasma concentration profiles provided by single-dose Phenylephrine HCl 20 mg GR systems of 6-hr and 9-hr zero-order release (GRZ6 & GRZ9), IR, and Rhinopront®.

### B. Multiple Dose

FIG. 8C shows a comparison of simulated plasma concentration profiles provided by 20 mg Phenylephrine HCl GR 6-hr square-root of time release (GRS6) and 20 mg Rhinopront® BID, and 40 mg GRS6 QD.

FIG. 8D illustrates a comparison of simulated plasma concentration profiles provided by 20 mg Phenylephrine HCl GR 9-hr square-root of time release (GRS9) and 20 mg Rhinopront® BID, and 40 mg GRS9 QD.

FIG. 8E shows a comparison of simulated plasma concentration profiles provided by 20 mg Phenylephrine HCl GR 6-hr zero-order release (GRZ6) and 20 mg Rhinopront® BID, and 40 mg GRZ6 QD.

FIG. 8F depicts a comparison of simulated plasma concentration profiles provided by 20 mg Phenylephrine HCl GR 9-hr zero-order release (GRZ9) and 20 mg Rhinopront® BID, and 40 mg GRZ9 QD.

FIG. 8G shows a comparison of simulated plasma concentration profiles provided by Phenylephrine HCl 30 mg GR systems of 6-hr and 9-hr zero-order release bid (q12 h) and IR 10 mg q4 h.

FIG. 8H illustrates a comparison of simulated plasma concentration profiles provided by Phenylephrine HCl 30 mg GR systems of 6-hr and 9-hr square-root of time release bid (q12 h) and IR 10 mg q4 h.

1. A gastric retentive dosage form, comprising:
an immediate release (IR) layer comprising a first dose of phenylephrine or a pharmaceutically acceptable salt thereof,
a gastric retentive (GR) layer comprising a second dose of phenylephrine or a pharmaceutically acceptable salt thereof dispersed in a polymeric matrix wherein the polymeric matrix is comprised of at least one polymer that upon imbibition of fluid swells to a size sufficient for gastric retention, and
wherein the second dose of phenylephrine is released over a time period of about 8 to 10 hours in vitro.

2. The dosage form of claim 1, wherein the dosage form is a tablet with a total weight of between about 400 mg to about 1000 mg.

3. The dosage form of claim 1, wherein the polymeric matrix comprises a polymer selected from the group consisting of poly(ethylene oxide), cellulose, alkyl-substituted celluloses, crosslinked polyacrylic acids, and xanthum gum.

4. The dosage form of claim 1, wherein the polymeric matrix comprises an alkyl-substituted cellulose.

5. The dosage form of claim 1, wherein the polymeric matrix comprises an alkyl-substituted cellulose selected from the group consisting of hydroxymethyl-cellulose, hydroxyethyl-cellulose, hydroxypropyl-cellulose, hydroxypropylmethyl-cellulose, and carboxymethyl-cellulose.

6. The dosage form of claim 1, wherein the polymeric matrix comprises hydroxypropylmethyl-cellulose having a viscosity ranging from 11,000 to 110,000 centipoise as measured in a 2% solution at 20° C.

7. The dosage form of claim 1, wherein the at least one polymer is poly(ethylene oxide) or hydroxypropyl methylcellulose.

8. The dosage form of claim 1, wherein the at least one polymer is a poly(ethylene oxide) having an average molecular weight of about 200,000 Daltons to about 12,000,000 Daltons.

9. The dosage form a claim 1, further comprising an anti-oxidant.

10. A method for making a tablet for the immediate release and extended release of phenylephrine, wherein the method comprises dry-blending phenylephrine HCl with at least one excipient.

11. The method of claim 10, further comprising pressing the dry-blend mixture to make a tablet and coating the tablet with an immediate release coating comprising a second drug.

12. The method of claim 11, wherein the second drug is phenylephrine.
